(19) United States
(12) Patent Application Publication
(10) Pub. No.: US 2021/0181188 A1
(43) Pub. Date: Jun. 17, 2021

(54) MHC-II GENOTYPE RESTRICTS THE ONCOGENIC MUTATIONAL LANDSCAPE

(71) Applicant: The Regents of the University of California, Oakland, CA (US)

(72) Inventors: Hannah Carter, San Diego, CA (US); Rachel Marty, San Diego, CA (US); Maurizio Zanetti, La Jolla, CA (US); Wesley Kurt Thompson, Arcadia, CA (US); Joan Font-Burgada, San Diego, CA (US)

(21) Appl. No.: 17/270,653

(22) PCT Filed: Aug. 23, 2019
(86) PCT No.: PCT/US2019/047981

§ 371 Date: Feb. 23, 2021

Related U.S. Application Data
(60) Provisional application No. 62/722,607, filed on Aug. 24, 2018.

(51) Int. Cl. G01N 33/53; C07K 7/00

(52) U.S. Cl. CPC G01N 33/5308; G01N 2333/70539; G01N 2800/50; C07K 7/00

(57) ABSTRACT

The present disclosure provides methods of determining the risk of a subject having or developing a cancer based on the affinity of the subjects MHC-II alleles for oncogenic mutations, methods for improving cancer diagnosis, and kits comprising agents that detect the oncogenic mutations in a subject.

### CROSS REFERENCE TO RELATED APPLICATIONS

This application claims the benefit of priority under 35 U.S.C. 119(e) to U.S. Application No. 62/722,607 filed Aug. 24, 2018.

### FEDERALLY SPONSORED RESEARCH OR DEVELOPMENT

This invention was made with government support under CA220009, OD017937, T15LM011271, DP5-OD017937, P41-GM103504, and 2015205295 awarded by the National Institutes of Health, the National Resource for Network Biology (NRNB), and the National Science Foundation. The government has certain rights in the invention.

### TECHNICAL FIELD

This disclosure generally relates to immunology.

### BACKGROUND

The Major Histocompatibility Complex (MHC) exposes protein content on the cell surface to allow detection of antigens by the immune system. This applies to non-self-antigens such as viral proteins as well as self-antigens such as tumor proteins.

Tumor cells harbor oncogenic alterations that can be presented to the immune system by the MHC, which normally causes immune recognition and elimination (sometimes referred to as “immune surveillance”). However, in order to grow, invade, and spread, tumors must evade immune surveillance. Common mechanisms of immune evasion include a) loss of the MHC molecules or b) the upregulation of immune checkpoint molecules on cell surfaces that normally regulate the amplitude and duration of a T cell response. Antibodies that block immune checkpoint molecules, known as immune checkpoint inhibitors (ICPi), can invigorate inactive and/or exhausted T cells, producing anti-tumor effects that confer long-term survival benefits in certain types of cancer. However, ICPi are effective in only 10-40% of patients for reasons that remain unclear. Meta-analyses of clinical trials in melanoma patients treated with ICPi suggest that young and female patients are characterized by low response rates. The reason(s) for the poor response of these two populations remains elusive, and developing a predictive assay would be beneficial.

### SUMMARY

Individual MHC genotype constrains the mutational landscape during tumorigenesis. Immune checkpoint inhibition reactivates immunity against tumors that escaped immune surveillance in approximately 30% of cases. Recent studies, however, demonstrated poorer response rates in female and younger melanoma patients. Although immune responses differ with sex and age, the role of MHC-based immune selection in this context is unknown. As described herein, female tumors accumulated more poorly presented driver mutations despite no sex-based differences in MHC genotype. Younger patients showed stronger effects of MHC-based driver mutation selection, with younger females showing compounded effects and nearly twice as much MHC-II based selection. This disclosure presents the first evidence that strength of immune selection during tumor development varies with sex and age, and may influence responsiveness to immune checkpoint inhibition therapy.

In one aspect, a computer implemented method for determining whether a subject is at risk of having or developing a cancer is provided. Such a method typically includes a) genotyping the subject's major histocompatibility complex class II (MHC-II); and b) scoring the ability of the subject's MHC-II to present a mutant cancer-associated peptide based upon a library of known cancer-associated peptide sequences sequences derived from subjects, wherein the produced score is the MHC-II presentation score. Generally, i) if the subject is a poor MHC-II presenter of specific mutant cancer-associated peptides, the subject has an increased likelihood of having or developing the cancer for which the specific mutant cancer-associated peptides are associated; or ii) if the subject is a good MHC-II presenter of specific mutant cancer-associated peptides, the subject has a decreased likelihood of having or developing the cancer for which the specific mutant cancer-associated peptides are associated.

Such a method can further include c) determining whether a biopsy sample obtained from the subject comprises DNA encoding a mutant cancer-associated peptide based upon a library of cancer-associated mutations obtained from subjects.

In some embodiments, the biopsy sample is a liquid biopsy sample. In some embodiments, the biopsy sample is a solid biopsy sample. Representative liquid biopsy samples include, without limitation, blood, saliva, urine, or other body fluid.

In some embodiments, the library of cancer-associated mutations is obtained by whole genome sequencing of subjects.

In some embodiments, the step of scoring the ability of the subject's MHC-II to present a mutant cancer-associated peptide comprises using a predicted MHC-II affinity for a given mutation xij, where x is the MHC-II affinity of subject i for mutation j to fit a mixed-effects logistic regression model that follows a model equation obtained from a large dataset of subjects from which MHC-II genotypes and presence of peptides of interest can be obtained:

logit(P(yᵢⱼ=1|xᵢⱼ))=ηⱼ+γ log(xᵢⱼ)

wherein: yᵢⱼ is a binary mutation matrix yᵢⱼ ∈{0,1} indicating whether a subject i has a mutation j; xᵢⱼ is a binary mutation matrix indicating predicted MHC-II binding affinity of subject i having mutation j; γ measures the effect of the log-affinities on the mutation probability; and ηj˜N(0, ϕ_{η}) are random effects capturing residue-specific effects, wherein the model tests the null hypothesis that γ=0 and calculates odds ratios for MHC-II affinity of a mutation and presence of a cancer.

In some embodiments, the predicted MHC-II affinity for a given mutation xᵢⱼ is a Subject Harmonic-mean Best Rank (PHBR) score. In some embodiments, the PHBR score is obtained by aggregating MHC-II binding affinities of a set of mutant cancer-associated peptides by referring to a pre-determined dataset of peptides binding to MHC-II molecules encoded by at least 12 different HLA alleles.

In some embodiments, the mutant cancer-associated peptide contains an amino acid substitution, and wherein the set of peptides consists of at least 15 of all possible 15-amino acid long peptides incorporating the substitution at every position along the peptide. In some embodiments, the mutant cancer-associated peptide contains an amino acid insertion or deletion, and wherein the set of peptides consists of at least 15 of all possible 15-amino acid long peptides incorporating the insertion or deletion at every position along the peptide. In some embodiments, the set of mutant cancer-associated peptides comprises any one or more of the mutations shown in Appendix A, wherein the presence of any one of these mutations indicates the presence of or increased risk of developing cancer.

Representative cancers include, without limitation, bladder urothelial carcinoma (BLCA), a breast invasive carcinoma (BRCA), a colon adenocarcinoma (COAD), a glioblastoma multiforme (GBM), a head and neck squamous cell carcinoma (HNSC), a brain lower grade glioma (LGG), a liver hepatocellular carcinoma (LIHC), a lung adenocarcinoma (LUAD), lung squamous cell carcinoma (LUSC), an ovarian serous cystadenocarcinoma (OV), a pancreatic adenocarcinoma (PAAD), a prostate adenocarcinoma (PRAD), a rectum adenocarcinoma (READ), a skin cutaneous melanoma (SKCM), a stomach adenocarcinoma (STAD), a thyroid carcinoma (THCA), a uterine corpus endometrial carcinoma (UCEC), or a uterine carcinosarcoma (UCS).

In another aspect, a computing system for determining whether a subject is at risk of having or developing a cancer is provided. Such a system typically includes a) a communication system for using a library of cancer-associated peptides derived from subjects; and b) a processor for scoring the ability of the subject's major histocompatibility complex class II (MHC-II) to present a mutant cancer-associated peptide based upon a library of cancer-associated peptides derived from subjects, wherein the produced score is the MHC-II presentation score.

In some embodiments, the step of scoring the ability of the subject's MHC-II to present a mutant cancer-associated peptide comprises using a predicted MHC-II affinity for a given mutation xij, where x is the MHC-II affinity of subject i for mutation j to fit a mixed-effects logistic regression model that follows a model equation obtained from a large dataset of subjects from which MHC-II genotypes and presence of peptides of interest can be obtained:

logit(P(yij=1|xij))=ηj+γ log(xij)

wherein: yij is a binary mutation matrix yij∈{,1} indicating whether a subject i has a mutation j; xij is a binary mutation matrix indicating predicted MHC-II binding affinity of subject i having mutation j; γ measures the effect of the log-affinities on the mutation probability; and ηj˜N(0, ϕη) are random effects capturing residue-specific effects, wherein the model tests the null hypothesis that γ=0 and calculates odds ratios for MHC-II affinity of a mutation and presence of a cancer.

In some embodiments, the predicted MHC-II affinity for a given mutation xij is a Subject Harmonic-mean Best Rank (PHBR)-II score. In some embodiments, the PHBR-II score is obtained by aggregating MHC-II binding affinities of a set of mutant cancer-associated peptides by referring to a pre-determined dataset of peptides binding to MHC-II molecules encoded by at least 12 different HLA alleles.

In some embodiments, the mutant cancer-associated peptide contains an amino acid substitution, and wherein the set of peptides consists of at least 15 of all possible 15-amino acid long peptides incorporating the substitution at every position along the peptide. In some embodiments, the mutant cancer-associated peptide contains an amino acid insertion or deletion, and wherein the set of peptides consists of at least 15 of all possible 15-amino acid long peptides incorporating the insertion or deletion at every position along the peptide.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

### Part A—Evolutionary Pressure Against MHC Class H Binding Cancer Mutations

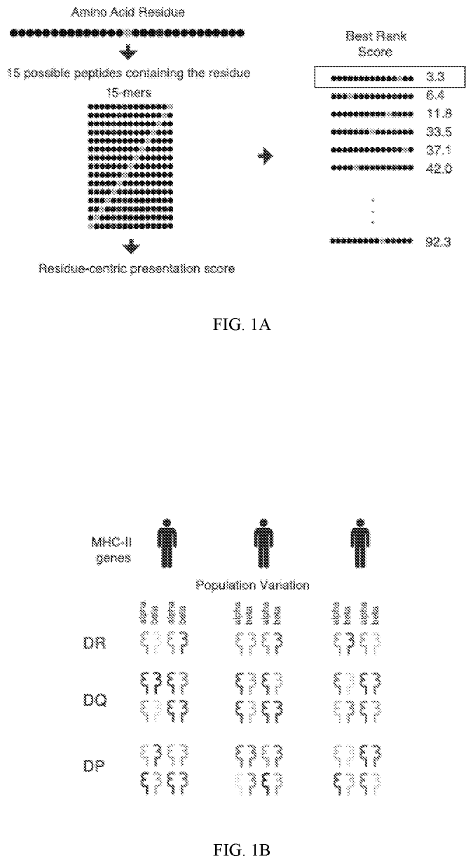

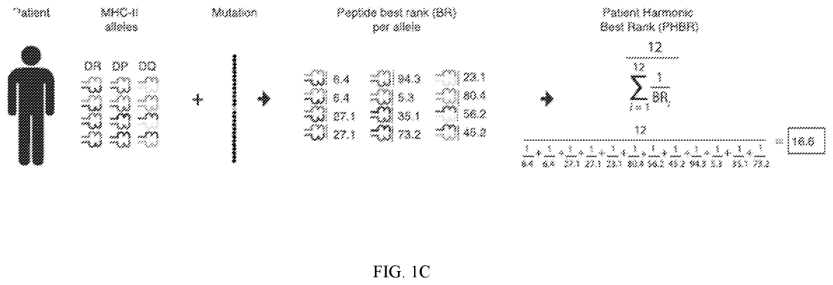

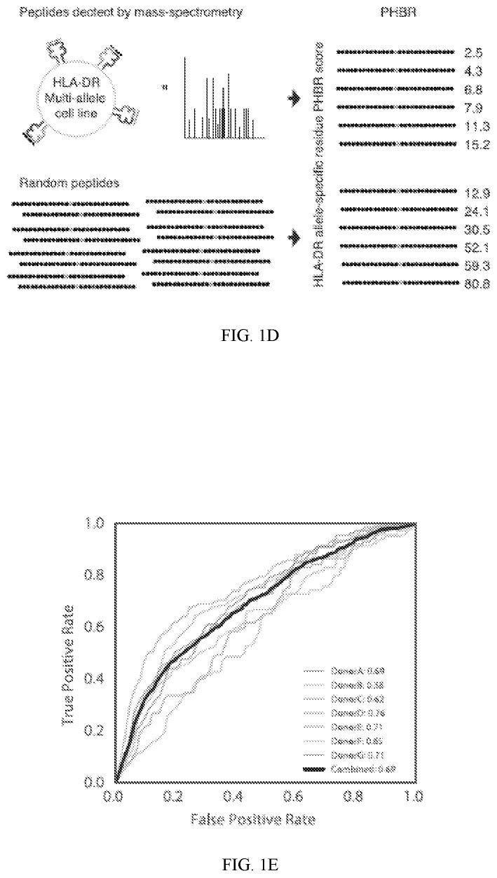

FIG. 1A-1E show the development of a residue-specific, patient-specific MHC-II presentation score. FIG. 1A-1C are schematic representations of the best rank (BR) presentation score for a residue (1A), MHC-II genetic diversity in the population (B), and the patient harmonic-mean best rank class II (PHBR-II) presentation score (1C). FIG. 1D shows an experimental schematic of the MS-based validation of the PHBR-II score. HLA-DR MS data from 7 donors was used to validate the PHBR-II score. FIG. 1E is a graph of ROC AUC curves showing the accuracy of the PHBR-II for classifying the extracellular presentation of a residue by a patient's HLA-DR genes for 7 donors (colors) and for all donors combined (black). The aggregated PHBR-II presentation scores for the 7 donors expressed HLA-DR alleles was compared to a set of random residues for the same HLA-DR alleles.

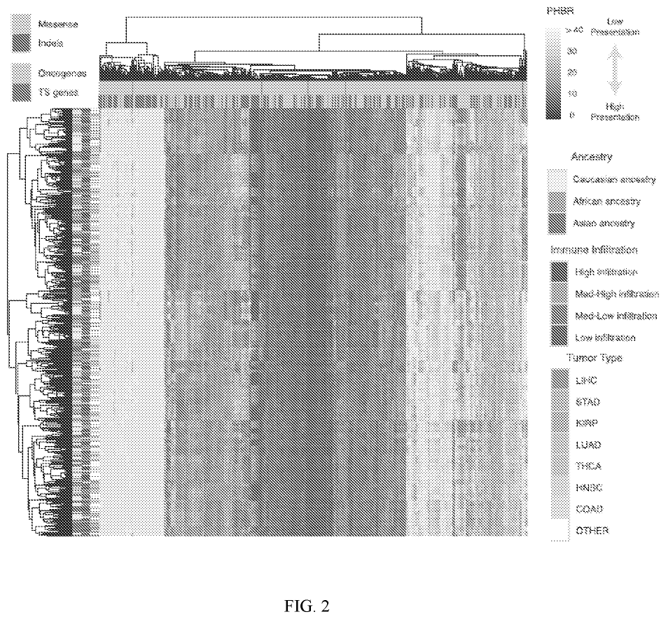

FIG. 2 is a pan-cancer overview of patient-mutation MHC-II presentation. A clustered heat map of patients in TCGA with the 1,018 frequent cancer mutations. Only 1,050 ancestry-distributed patients are included for spatial reasons. The heat map is colored by PHBR-II score. Column and row coloring highlight groupings of patients and mutations into different categories. TS, tumor suppressor.

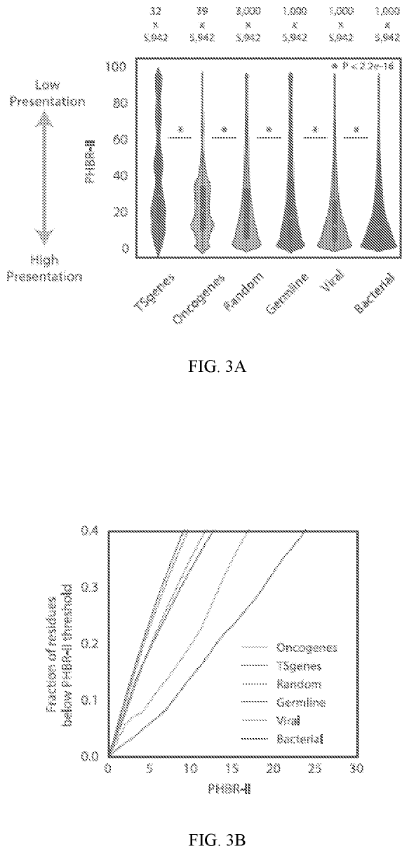

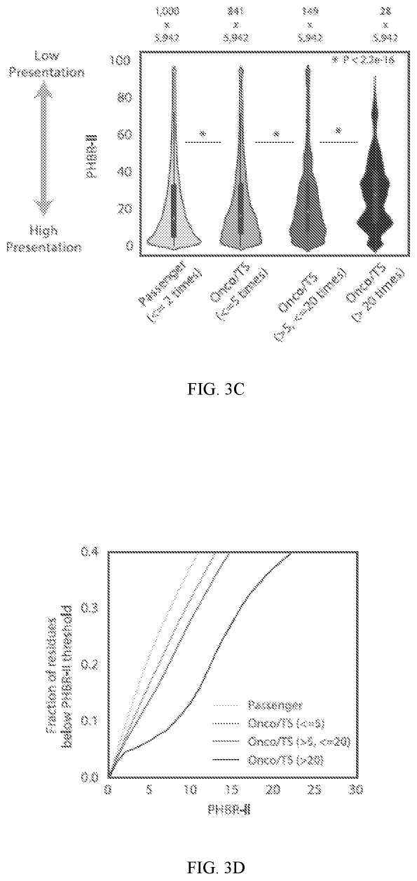

FIG. 3A is a violin plot denoting the distribution of PHBR-II presentation scores across all patients in TCGA for 6 different classes of residue. TS, tumor suppressor. Mutations observed >10 times in TCGA are displayed. The white dots represent the median, the thick dark gray lines denote the interquartile of the data, and the thin dark gray lines denote the 1.5 IQR range.

FIG. 3B shows the cumulative distribution functions (CDF) for the 6 different classes of residue.

FIG. 3C is a violin plot with the distribution of somatic mutations occurring at different frequencies: passenger mutations in non-cancer implicated genes observed <2 in TCGA, and mutations in cancer implicated genes observed 3-10 times, 11-40 times, and >40 times in TCGA. The white dots represent the median, the thick dark gray lines denote the interquartile of the data, and the thin dark gray lines denote the 1.5 IQR range.

FIG. 3D is a CDFs for somatic mutations occurring at different frequencies.

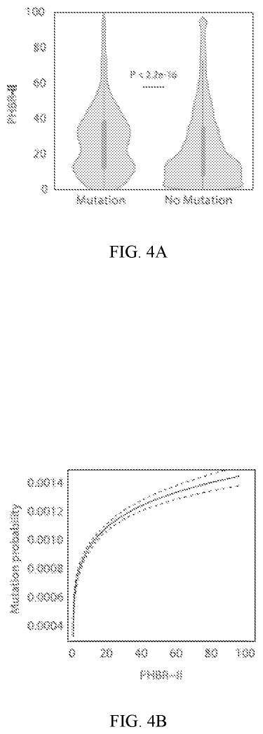

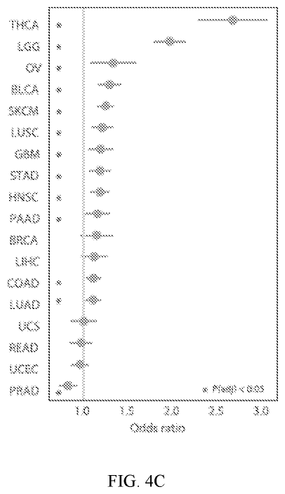

FIG. 4A is a violin plot denoting the difference in PHBR-II scores when the 5,942 patients are split by mutation occurrence, considering only mutations observed >2 times across tumors.

FIG. 4B shows nonparametric estimate of the logit-mutation probability as a function of PHBR-II scores considering mutations observed >2 times across tumors.

FIG. 4C shows the MHC-II ORs (gray circles) and 95% CIs (bars) associated with a 1-unit increase in log-PHBR-II score for different cancer types.

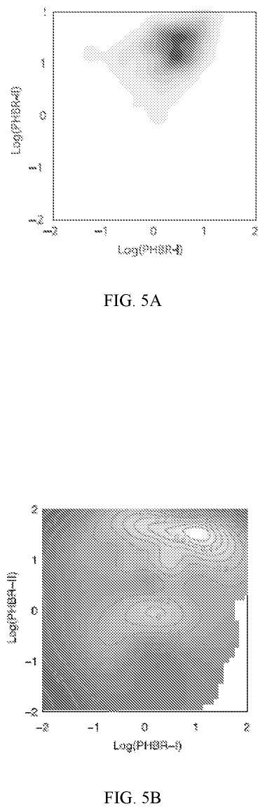

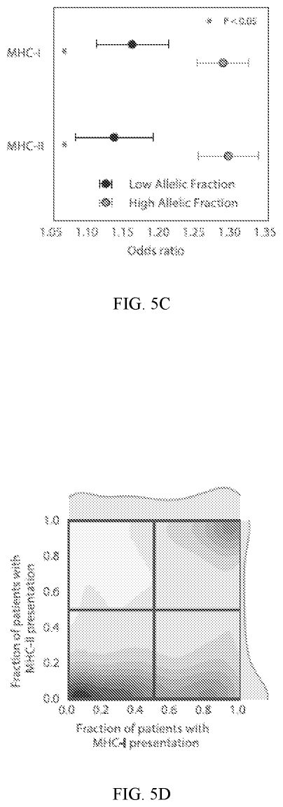

FIG. 5A is a kernel density plot with the density of PHBR-II and -I scores across cancer-driving mutations.

FIG. 5B is a heat map of mutation probability for all combinations of PHBR-II and -I scores. Dark red represents low probability and white represents high probability.

FIG. 5C shows the MHC-I and MHC-II ORs (gray circles) and 95% CIs associated with a 1-unit increase in log-PHBR-II score. Results are shown for mutations with low allelic fraction (dark gray) and high allelic fraction (light gray). Bars show 95% CIs.

FIG. 5D is a kernel density plot showing the density of mutations according to the fraction of patients who can present it with MHC-I and MHC-II. The red bars denote the four quadrants of the graph.

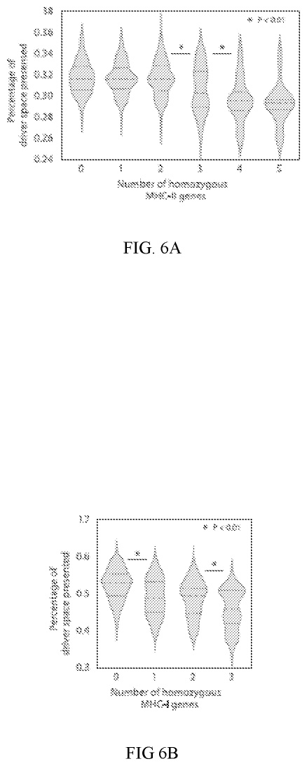

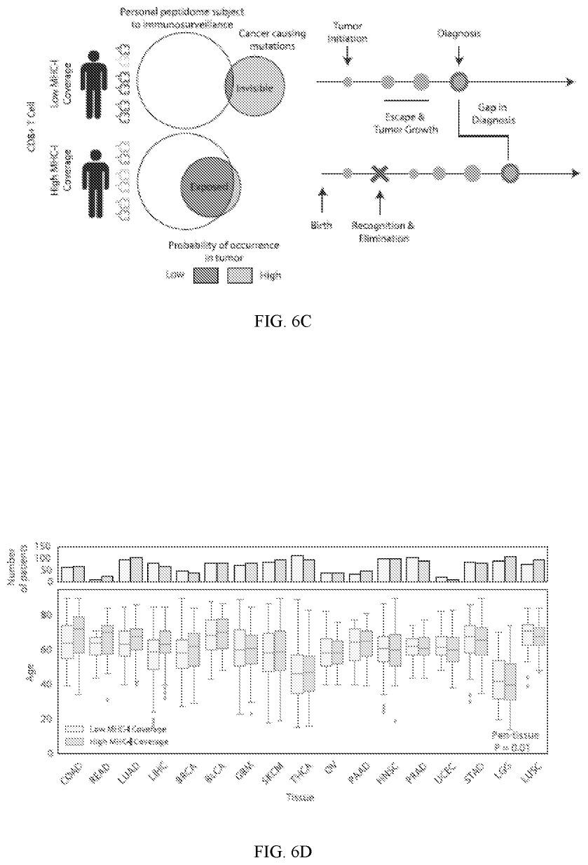

FIG. 6A is a violin plot depicting the distributions of the percentage of the 1,018 driver mutations presented by MHC-II for patients with varying numbers of homozygous genes.

FIG. 6B is a violin plot depicting the distributions of the percentage of the 1,018 driver mutations presented by MHC-I for patients with varying numbers of homozygous genes.

FIG. 6C is a schematic showing the effect of MHC coverage on age at diagnosis.

FIG. 6D is a box plot of the distributions of age at diagnosis for patients separated by tumor type and percentage of the driver space presented for MHC-I. Bars indicate the 1.5 interquartile range.

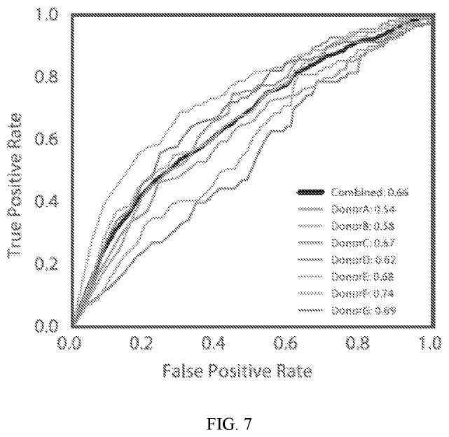

FIG. 7 is a graph showing the development of a residue-specific, patient-specific MHC-II presentation score. ROC AUC curves showing the accuracy of the PHBR-II including peptides of length 13-25 for classifying the extracellular presentation of a residue by a patient's HLA-DR genes for 7 donors (colors) and for all donors combined (black). The aggregated PHBR-II presentation scores for the 7 donors expression HLADR alleles was compared to a set of random residues for the same HLA-DR alleles.

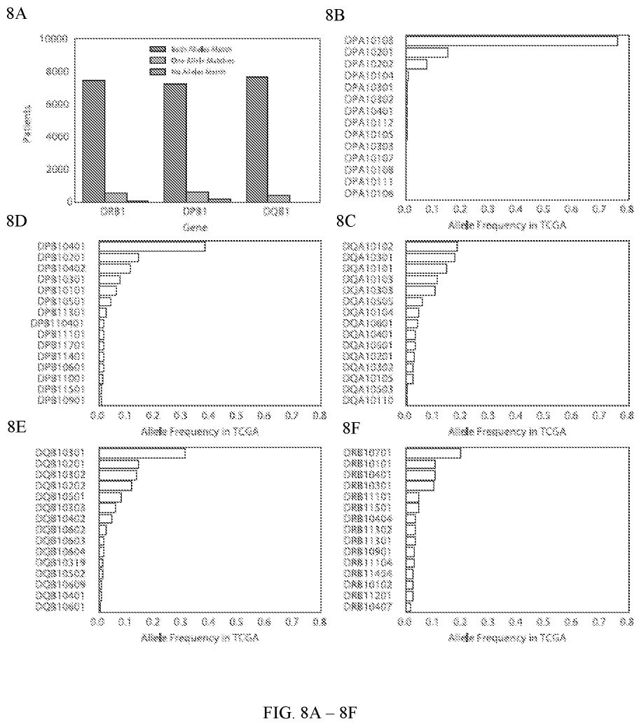

FIG. 8A is a graph showing the agreement of hla types for patients typed with HLA-HD and xHLA.

FIG. 8B is a graph showing the frequency of MHC-II alleles occurring in TCGA-HLA-DPA.

FIG. 8C is a graph showing the frequency of MHC-II alleles occurring in TCGA-HLA-DPB.

FIG. 8D is a graph showing the frequency of MHC-II alleles occurring in TCGA-HLA-DQA.

FIG. 8E is a graph showing the frequency of MHC-II alleles occurring in TCGA-HLA-DQB.

FIG. 8F is a graph showing the frequency of MHC-II alleles occurring in TCGA-HLA-DRB.

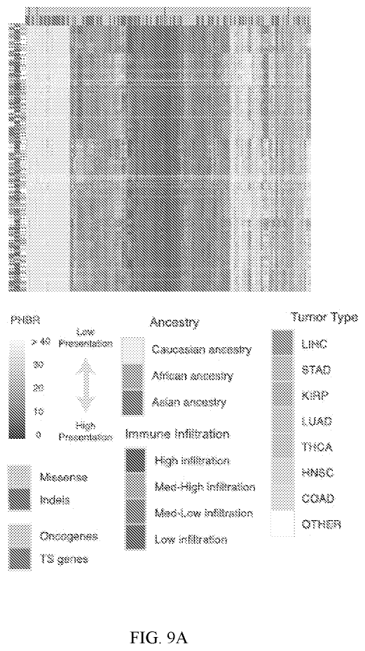

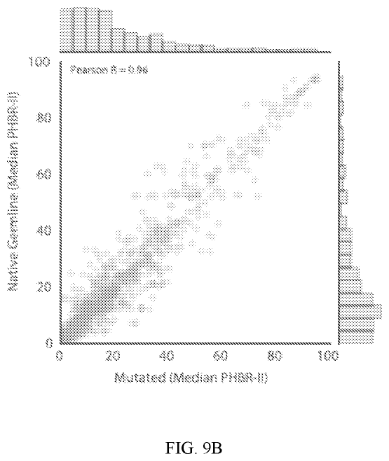

FIG. 9A is a clustered heat map of patients in TCGA with the native germline sequence 1,018 frequent cancer mutations. The same 1,050 patients are represented as in FIG. 2. The heat map is colored by PHBR-II score. Column and row coloring highlight groupings of patients and mutations into different categories.

FIG. 9B is a scatterplot showing the median population PHBR-II score for each of the 1,018 mutations and their native germline sequence.

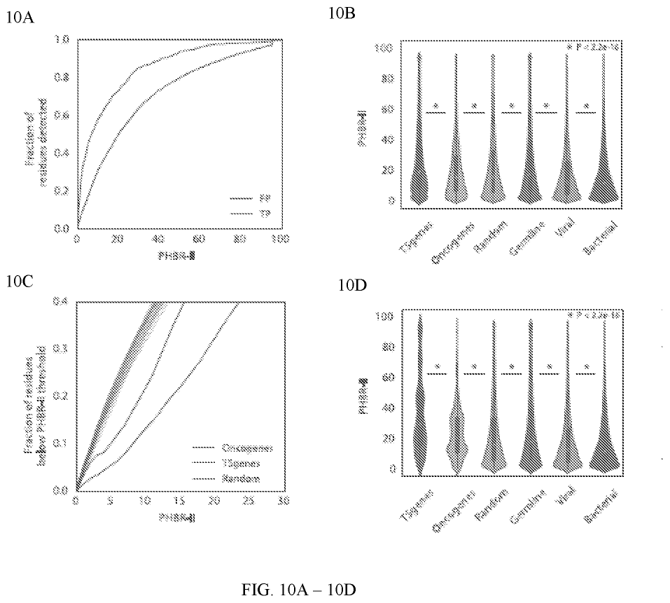

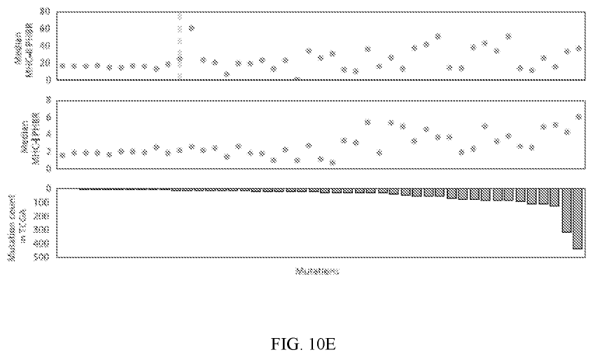

FIG. 10A shows the cumulative distribution functions denoting the fraction of true positive and false positive residues detected for each PHBR-II score in the mass spectrometry validation.

FIG. 10B shows a violin plot denoting the distribution of PHBR-II presentation scores across all TCGA patients for 6 different classes of residue. Cancer mutations observed >2 times in TCGA are displayed. White dots represent the median.

FIG. 10C shows the cumulative distribution of 20 sets of random 1,000 mutations. Shown alongside the cumulative distribution from oncogenes and tumor suppressor genes.

FIG. 10D shows a violin plot denoting the distribution of PHBR-II presentation scores across non-cancer dbGaP patients for 6 different classes of residue. White dots represent the median.

FIG. 10E shows two dot plots showing the median PHBR-II and -I presentation scores for all 5,942 patients of the 1,018 recurrent cancer mutations grouped by their mutation count in TCGA and displayed as a median. The number of times the mutation group is observed in TCGA is plotted in the bottom panel. The light gray line highlights the mutations observed 10 times.

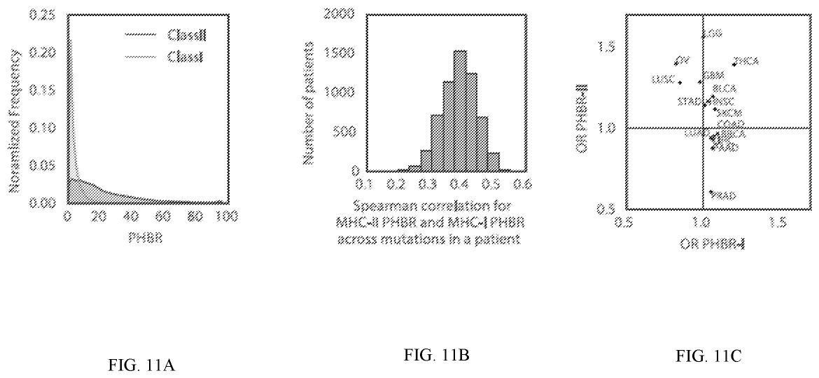

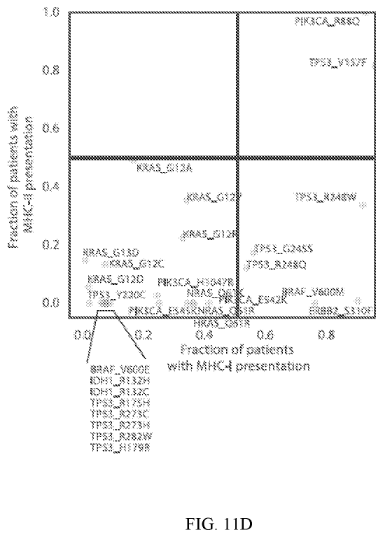

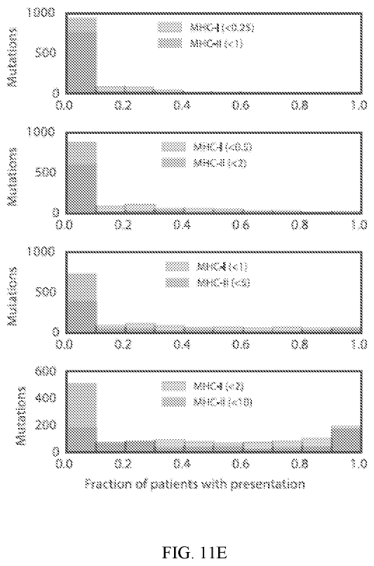

FIG. 11A shows the distribution of PHBR-II and PHBR-I scores.

FIG. 11B shows the distribution of spearman rho correlations for PHBR-II and PHBR-I scores across all driver mutations for every patient in TCGA.

FIG. 11C is a scatterplot showing the relationship between tissue specific ORs for MHC-II and MHC-I with a joint model for tumor types with at least 100 patients.

FIG. 11D is a scatterplot showing mutations observed at least 20 times in TCGA. Each point is placed according to the fraction of patients who can present it with MHC-I and MHC-II.

FIG. 11E are histograms showing the variation in the number of mutations with different fractions of presentation by both MHC-I and MHC-II across several presentation thresholds.

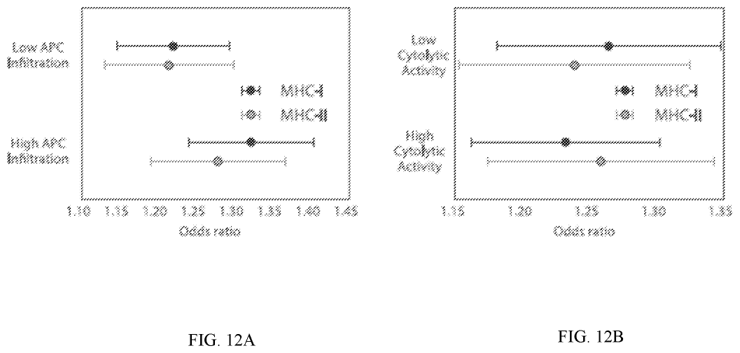

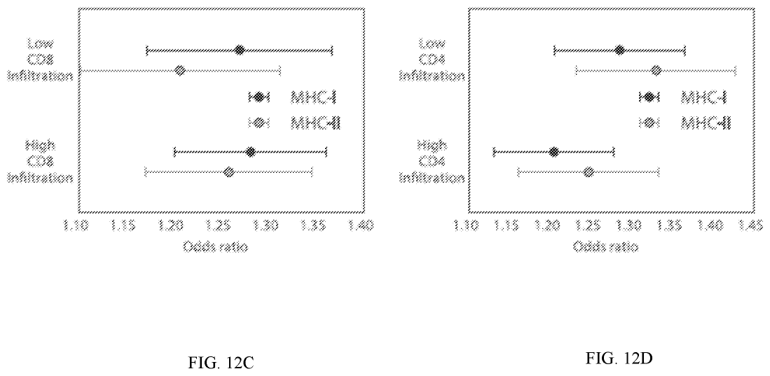

FIG. 12A-12D shows MHC-based mutation selection for differing levels of immune activity. The MHC-I and MHC-II ORs (circles) and 95% CIs (bars) associated with a 1-unit increase in log-PHBR-II score. The results are shown for patients with low and high (S6A) APC infiltration, (S6B) cytolytic activity, (S6C) CD8+ T cell infiltration and (S6D) CD4+ T cell infiltration.

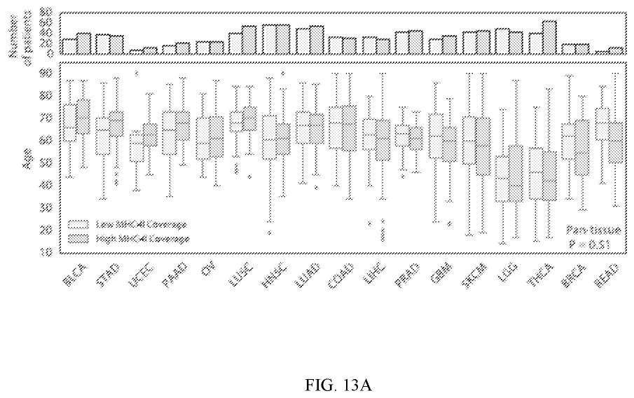

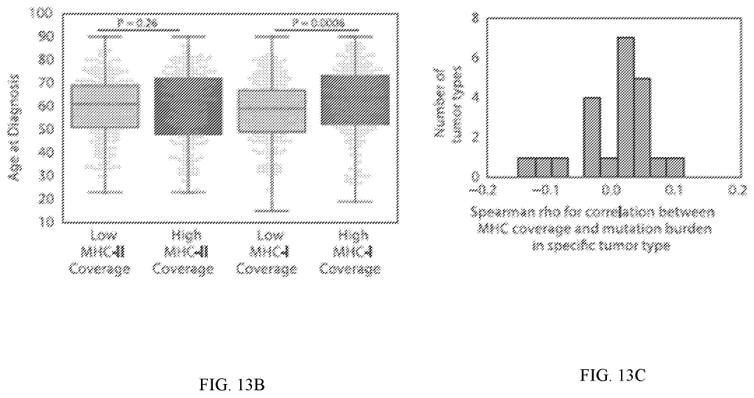

FIG. 13A is a box plot denoting the distributions of age at diagnosis for patients separated by tumor type and percentage of the driver space presented for MHC-II. The number of patients in each category is visualized above with a bar plot. Bars indicate the 1.5 interquartile range.

FIG. 13B is a box plots showing the age at diagnosis for patients with extreme 5% of patients for MHC-I and MHC-II coverage. Bars indicate the 1.5 interquartile range.

FIG. 13C is a histogram representing the spearman rho correlations for each tumor type between MHC-I coverage and mutation burden.

Part B—Strength of Immune Selection in Tumors Varies with Sex and Age

FIG. 14A-14D are graphs showing sex- and age-specific MHC presentation of observed, expressed driver mutations. FIGS. 1A-1B are box plots denoting the distribution of PHBR-I (1A) and PHBR-II (1B) scores for expressed driver mutations in female and male pan-cancer patients. FIGS. 1C-1D are box plots denoting the distribution of PHBR-I (1C) and PHBR-II (1D) scores for expressed driver mutations in younger and older pan-cancer patients.

FIG. 15A-15B are graphs showing the integrated sex- and age-specific analysis of PHBR-I (2A) and PHBR-II (2B) scores for the observed driver mutations in pan-cancer integrated sex- and age-specific patient cohorts.

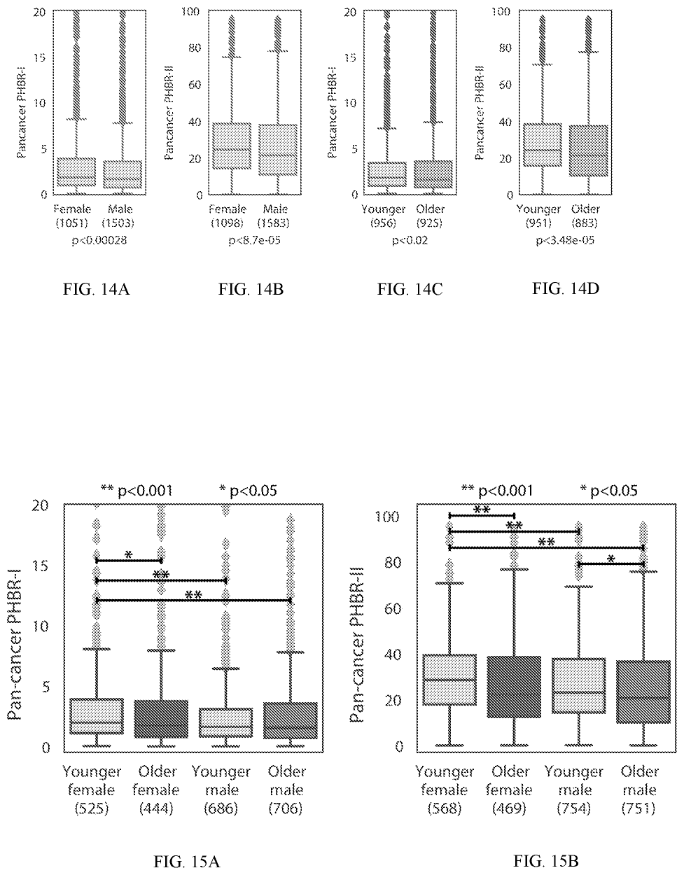

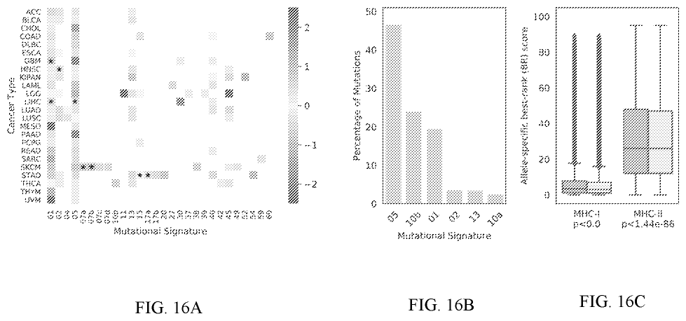

FIG. 16A shows the log 2 male (blue) to female (pink) ratios of mutational signatures for each tumor type.

FIG. 16B shows the percentage of mutations in the set of driver mutations that are part of each mutational signature.

FIG. 16C is a box plot comparing allele-specific MHC-I and MHC-II presentation scores of C>T or T>C driver mutations (green) versus driver mutations resulting from other base substitutions (yellow).

FIGS. 17A and 17B are box plots denoting the distribution of PHBR-I (4A) and PHBR-II (4B) scores for driver mutations in female and male pan-cancer patients.

FIGS. 17C and 17D are box plots denoting the distribution of PHBR-I (4C) and PHBR-II (4D) scores for driver mutations in younger and older pan-cancer patients.

FIGS. 17E and 17F are box plots denoting the distribution of PHBR-I (4E) and PHBR-II (4F) scores for driver mutations among integrated sex- and age-specific pan-cancer patient cohorts.

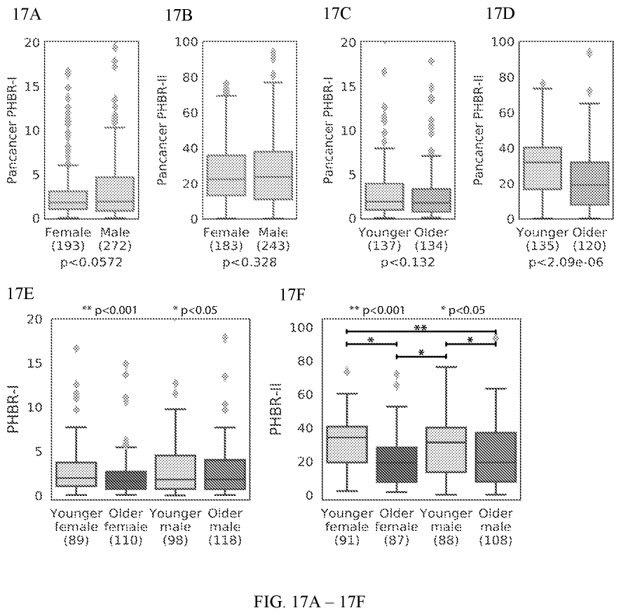

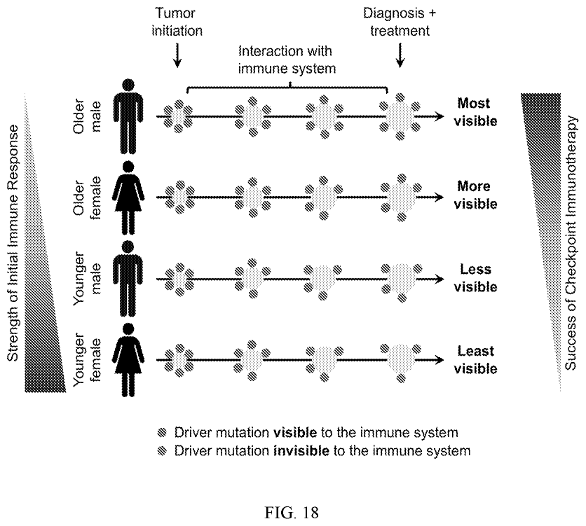

FIG. 18 is a schematic of a proposed model of the relationship between immune selection and immunotherapy in cancer patients. Young females experience the strongest immune response, rendering their diagnosed tumors very invisible to the immune system and difficult to treat with ICPi. On the other end of the spectrum, old males experience the weakest immune response, leaving their diagnosed tumors very visible to the immune system and open to attack when stimulated with ICPi.

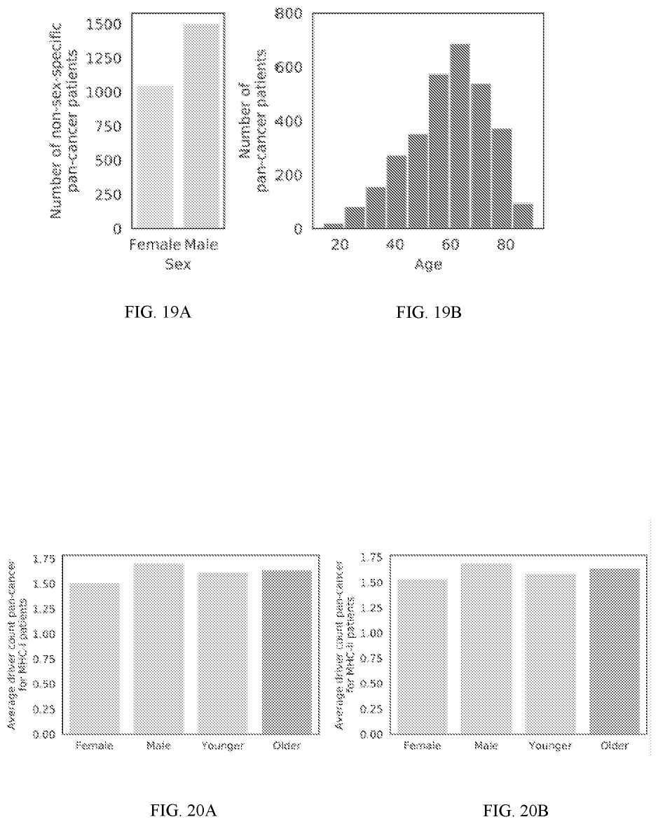

FIG. 19A is a bar plot denoting the number of male and female patients in the pan-cancer cohort with sex-specific cancers (BRCA, CESC, OV, PRAD, TGCT, UCEC, UCS) removed.

FIG. 19B is a histogram denoting the distribution of ages when patients were diagnosed with cancer in the pan-cancer cohort. Sex-specific cancers mentioned previously were retained for age analyses.

FIG. 20A-20B are bar plots denoting the average number of driver mutations in each sex- and age-specific cohort for (20A) patients with confident MHC-I calls, and (20B) patients with confident MHC-II calls.

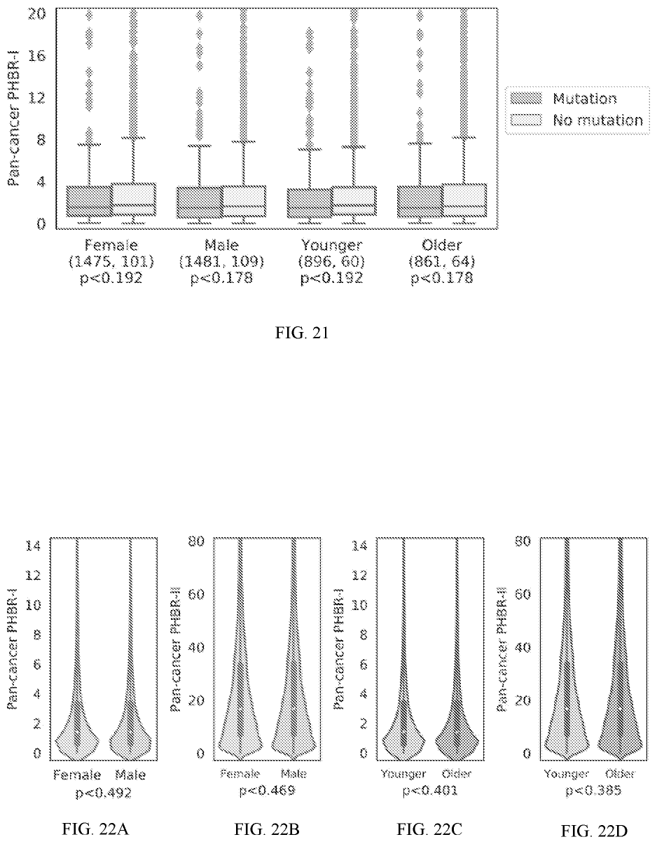

FIG. 21 is a sex- and age-specific MHC presentation of common driver mutations for patients with and without MHC-I mutations. Box plots denoting the distribution of PHBR-I scores for expressed driver mutations in female, male, younger, and older pan-cancer patients with and without MHC-I mutations. The average number of driver mutations pan-cancer per cohort. Bar plots denoting the average number of driver mutations in each sex- and age-specific cohort for patients with confident MHC-II calls.

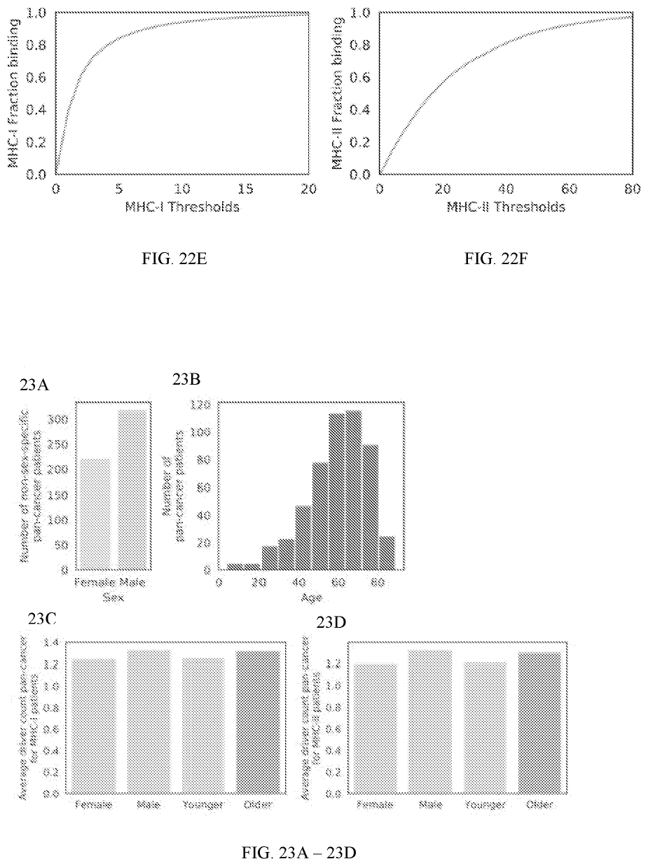

FIG. 22A-22F are graphs showing sex- and age-specific MHC presentation of common driver mutations. (22A-22D) Violin plots denoting the distribution of (22A, 22C) PHBR-I and (22B, 22D) PHBR-II scores across all common cancer driving mutations. (22E, 22F) The distribution of the fraction of all common cancer driving mutations that each patient can bind along various thresholds with (22E) MHC-I and (22F) MHC-II.

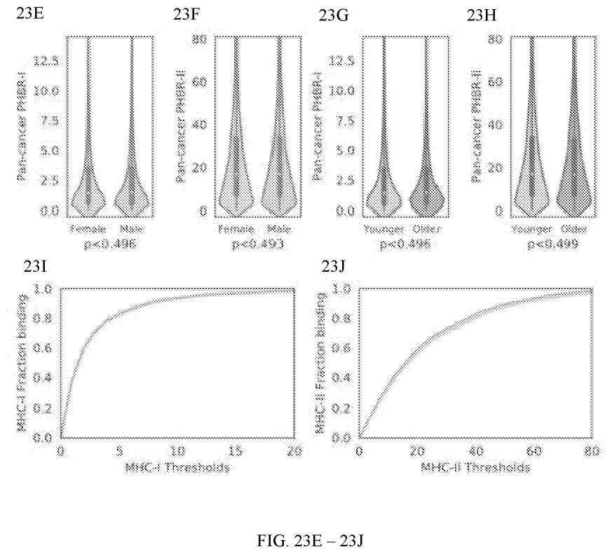

FIG. 23A-23J is data that provides an overview of the validation cohort. (23A) A bar plot denoting the number of male and female patients in the pan-cancer validation cohort. (23B) A histogram denoting the distribution of ages when patients were diagnosed with cancer in the pan-cancer validation cohort. (23C-23D) Bar plots denoting the average number of driver mutations in each sex- and age-specific cohort for (23C) patients with MHC-I calls, and (23D) patients with MHC-II calls. (23E-23H) Violin plots denoting the distribution of (23E, 23G) PHBR-I and (23F, 23H) PHBR-II scores across all common cancer driving mutations. (231, 23J) The distribution of the fraction of all common cancer driving mutations that each patient can bind along various thresholds with (231) MHC-I and (23J) MHC-II.

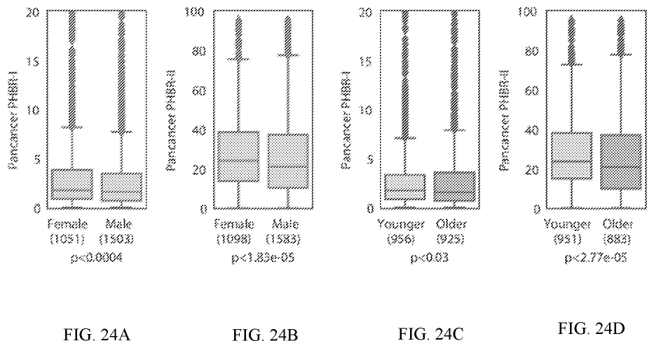

FIG. 24A-24D are graphs showing sex- and age-specific MHC presentation of observed mutations, without expression confirmation. (A24-24B) Box plots denoting the distribution of (24A) PHBR-I and (24B) PHBR-II scores for driver mutations in female and male pan-cancer patients. (24C-24D) Box plots denoting the distribution of (24C) PHBR-I and (24D) PHBR-II scores for driver mutations in younger and older pan-cancer patients.

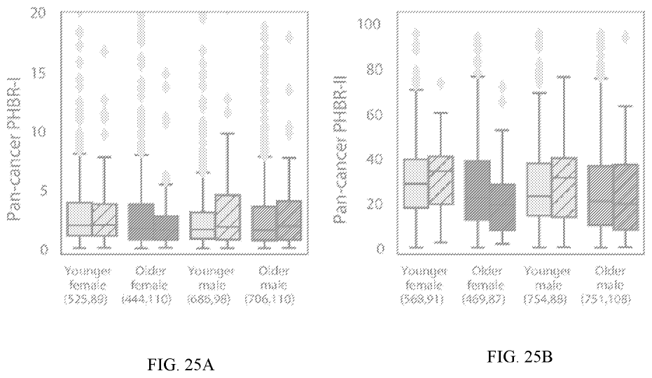

FIG. 25A-25B are graphs comparing driver mutation presentation by MHC between discovery (plain) and validation (striped) cohorts stratified by age and sex. (25A) PHBR-I and (25B) PHBR-II score distributions for the observed driver mutations in each cohort are compared across sex- and age-matched patient groups, with both discovery and validation cohorts using 52 and 68 for younger and older age thresholds, respectively.

### DETAILED DESCRIPTION

MHC-II molecules typically present 12-16 amino acid peptides to CD4+ T cells. CD4+ T cells play a more complex role than CD8+ T cells. While possessing cytotoxic effector properties similar to CD8+ T cells, CD4+ T cells also exert a wide range of regulatory functions that distinguish them from CD8+ T cells. Classically, CD4+ T cells provide functional help to B cells, CD8+ T cells, and CD4+ T cells in the form of cooperation involving cognate interaction with an antigen presenting cell (B cell or dendritic cell). The role of CD4+ T cells in tumor immunity and protection has been demonstrated in the mouse, and patients responding to immunotherapy show a strong proliferative CD4+ T cell response to tumor-associated antigens. In addition, adoptive CD4+ T cell therapy has been associated with durable clinical responses in melanoma and cholangiocarcinoma patients.

Early detection, diagnosis, and treatment of tumors is a major determinant of patient morbidity and mortality. Accurate predictions of when, where, and how tumors are likely to arise would have enormous implications for cancer screening and could improve survival rates. While the main contributor to the development of most adulthood tumors is sporadic somatic mutation, germline variants have been implicated as a determinant of tumor characteristics. Here, we propose that the MHC-II genotype is an additional such germline influence.

This disclosure describes the essential role of MHC-II molecules in antigen presentation and in immune detection of mature tumors through neoantigen recognition. MHC-II, like MHC-I, is highly variable among humans, with 4,802 documented alleles. However, the antigen affinity of each MHC-II molecule is influenced by two genes, producing a combinatorial effect that leads to higher variation than MHC-I. In addition, the average MHC binding affinity for MHC-II-restricted peptides required to activate CD4+ T cells is less stringent than that for MHC-I restricted peptides, the MHC-II peptide binding groove structure allows more promiscuous binding of peptides, and CD4+ T cell responses can extend to encompass additional antigens after initial activation (epitope spreading). As described herein, however, we surprisingly found that MHC-II genotype has an even stronger influence over mutation probability than does the MHC-I genotype.

MHC-II appears to exert a stronger selective pressure than MHC-I, leading to a stronger effect by MHC-II on somatic mutation probability. This role aligns with the understanding of CD4+ T cells as a necessary component of the activation and regulation of CD8+ T cells. While the diversity of an individual's MHC-I may play a role in tumor susceptibility, MHC-I appears to have weaker effects on mutation selection.

Notably, as described herein, MHC-II had stronger effects than MHC-I in shaping the driver mutations of a tumor. Interestingly, these effects appear to be less patient-specific than MHC-I, perhaps due to the promiscuous nature of MHC-II peptide binding. Furthermore, these effects could be driven by a faster evasion of MHC-I presentation than MHC-II presentation due to mechanisms like HLA mutation or HLA loss of heterozygosity that would occur within the tumor but are unlikely to affect the MHC-II on professional APCs. Another possibility is that MHC-II presentation and CD4+ T cell recognition may be a necessary prerequisite to CD8+ T cell cytotoxicity and tumor elimination, in agreement with the regulatory role of CD4+ T cells. We reason that the stronger effect of MHC-II on the odds of acquiring a mutation is consistent with a dual regulatory and effector CD4+ role. If the role of CD4+ T cells was purely regulatory, MHC-I specificity would be expected to drive mutation probability. Therefore, the role of the MHC-II genotype and MHC-II presentation needs to be properly weighted to understand the role of the interplay between mutational burden and tumor evolution. This understanding will be essential in the development of immunotherapies, likely being a critical component of their future success.

This disclosure indicates that the response rate to immune checkpoint inhibitors (ICPi) may be dependent on the strength of immune selection occurring early in tumorigenesis. Methods to accurately predict the impact of immunoediting on a patient-specific basis may lead to better predictive algorithms for response to therapy. As a corollary, we posit that ICPi treatment is likely to have a reduced effect in younger female patients since this treatment will attempt to reactivate T cells for immunologically invisible neoantigens. Rather, adaptive T cell therapy against patient-validated neoantigens or therapeutic vaccination against conserved antigens will likely be more beneficial in these patients. Finally, these findings shed new light on the role of immune surveillance in cancer progression.

As described herein, we found that predicted MHC-II presentation of cancer-related somatic mutations shape tumor development through variation in antigen presentation in complementary fashion to MHC-I, highlighting the need to consider the independent, yet complementary, roles of CD4+ and CD8+ T cells in the selection and elimination of tumors.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Part A—Evolutionary Pressure Against MHC Class H Binding Cancer Mutations

### Example 1—Data Acquisition

Data were obtained from publicly available sources including The Cancer Genome Atlas (TCGA) Research Network (cancergenome.nih.gov/ on the World Wide Web), The Allele Frequency Net Database (Gonzalez-Galarza et al., 2018, Methods Mol. Biol., 1802:49-62), Ensembl, Exome Variant Server, UniProt (UniProt Consortium, 2015), or cited literature (Ciudad et al., 2017, J. Leukoc. Biol., 101:15-27). TCGA normal exome sequences and TCGA clinical data were also downloaded from the GDC. Furthermore, TCGA somatic mutations were accessed from the NCI Genomic Data Commons (portal.gdc.cancer.gov/ on the World Wide Web). Population level HLA frequencies were obtained from the Allele Frequency Net Database. Common germline variants were downloaded from the Exome Variant Server NHLBI GO Exome Sequencing Project (ESP), Seattle, Wash. Finally, viral and bacterial peptides were obtained from UniProt.

### Example 2—Single Allele Presentation Score Construction

To create a residue-centric presentation score, we evaluated allele-based ranks for peptides containing the residue of interest. Each allele-based rank was predicted using the NetMHCIIPan-3.1 tool, downloaded from the Center for Biological Sequence Analysis (Karosiene et al., 2013, Immunogenetics, 65:711-724). NetMHCIIPan-3.1 takes a peptide and an MHC-II protein (HLA-DRB1, HLA-DPA1/DPB1 or HLA-DQA1/DQB1) and returns binding affinity IC50 scores and corresponding allele-based ranks. Peptides with rank <10 and <2 are considered to be weak and strong binders, respectively. Allele-based ranks were used to represent peptide binding affinity. We previously established the best rank of possible peptides containing the residue as an effective estimator of extracellular presentation (Marty et al., 2017, Cell, 171:1272-83). Here, we evaluated two approaches to selecting the set of peptides containing the residue to consider:
All 15-mers: Every peptide of length 15 containing the residue of interest, totaling 15 peptides.
13-mers through 25-mers: Every peptide of length 13 through length 25 containing the peptide, totaling in 247 peptides (Wieczorek et al., 2017, Front. Immunol., 8:292).

Insertion and deletion mutations were modeled by the resulting peptides that differed from the native sequence and tested with the same peptide-set parameters. These two peptide selection models were compared based on performance in a multi-allelic setting and the all 15-mers model was selected (see below).

### Example 3—Multi-Allele Presentation Score Construction

We defined a patient presentation score to represent a particular patient's ability to present a residue given their distinct set of 12 HLA-encoded MHC-II molecules (4 combinations of HLA-DPA1/DPB1 and HLA-DQA1/DQB1; 2 alleles of HLA-DRB1 considered twice each (since HLA-DRA1 is invariant) for consistency between resulting molecules). The Patient Harmonic-mean Best Rank (PHBR) score was assigned as the harmonic mean of the best residue presentation scores for each of the 12 MHC-II molecules. A lower patient presentation score indicates that the patient's MHC-II molecules are more likely to present a residue on the cell surface.

### Example 4—Mass Spectrometry-Based Presentation Score Validation

In order to test the performance of the different peptide sets that could compose the multi-allelic PHBR score to predict presentation, we used published MS data for 7 cell lines expressing 2-3 HLA-DRB1 alleles typed to the fourth digit (Ciudad et al., 2017, J. Leukoc. Biol., 101:15-27). Ciudad et al. (2017, J. Leukoc. Biol., 101:15-27) catalogs peptides observed in complex with MHC-II (HLA-DR) on the cell surface for 7 different combinations of 2-3 HLA-DRB1 alleles, with 70 to 240 mappable peptides each. These data were combined with a set of random peptides to construct a benchmark for evaluating the performance of scoring schemes for identifying residues presented on the cell surface as follows:
Converting MS peptide data to residues: the Ciudad et al. (2017, J. Leukoc. Biol., 101:15-27) MS data provides peptides observed in complex with the MHC-II, whereas our presentation score is residue-centric. For each peptide in the MS data, we selected the residue at the center (or one residue before the center, in the case of peptides of even length) as the residue for calculating the residue-centric presentation score.
Selection of background peptides: we selected 3000 residues at random from the Ensembl human protein database (Release 89) (Aken et al., 2017, Nuc. Acids Res., 45(D1):D635-42) to ensure balanced representation of MS-bound and random residues. The randomly selected residues represent an approximation of a true negative set of residues that would likely not be presented on the cell surface. If this assumption is flawed, the resulting AUC will underestimate the true accuracy.
Scoring benchmark set residues: we calculated PHBR presentation scores with each peptide set for all of the selected residues from the Ciudad et al. (2017, J. Leukoc. Biol., 101:15-27) data and the 3000 random residues against each of the 7 cell lines.
Evaluating scoring scheme performance using the benchmark: for each scoring scheme, scores were calculated for each cell line and pooled across the 7 cell lines. We plotted and compared ROC curves for each score formulation by calculating the True Positive Rate (% of observed MS residues predicted to bind at a given threshold) and the False Positive Rate (% of random residues predicted to bind at a given threshold) from 0 to 100 with steps of 0.5. Finally, we assessed overall score performance using the area under the curve (AUC) statistic. Based on this analysis, the 15-mer peptide set was used to construct the PHBR presentation score for all subsequent analyses.

### Example 5—HLA-II Typing

HLA genotyping was performed for genes HLA-DRB1, HLA-DPA1, HLA-DPB1, HLA-DQA1 and HLA-DQB1, which encode three protein determinants of MHC-I peptide binding specificity, HLA-DR, HLA-DP, and HLA-DQ. TCGA samples (see Table 51 in doi.org/10.1016/j.cell.2018.08.048 on the World Wide Web) were typed with HLA-HD (Kawaguchi et al., 2017, Hum. Mutat. 38:788-97), using default parameters. HLA-HD requires germline (whole blood or tissue matched) whole exome sequenced samples. The tool reports 100% 4-digit validation accuracy across 90 low-coverage exomes. Samples with very low coverage on specific genes are left untyped by HLA-HD. Patients were assigned an HLA-DR type if they were successfully typed for HLA-DRB1. Patients were assigned HLA-DP and -DQ types if they had successful typing for HLA-DPA1/HLA-DPB1 and HLA-DQA1/HLA-DQB1, respectively. Samples were validated by xHLA (Xie et al., 2017, PNAS USA, 114:8059-64), run with default parameters, and only patients where all alleles agreed were included in the analysis (FIG. 8A; see Table 51 in doi.org/10.1016/j.cell.2018.08.048 on the World Wide Web). Allele frequencies were visualized with horizontal bar graphs (FIGS. 8B-8F).

### Example 6—Selection of Recurrent Oncogenic Mutations, Passenger-Like and Non-Driver Mutations

Somatic mutations were considered to be recurrent and oncogenic if they occurred in one of the 100 most highly ranked oncogenes or tumor suppressors described by Davoli et al. (2013, Cell, 155:948-62) and were observed in at least 3 TCGA samples. Among these, we retained only mutations that would result in predictable protein sequence changes that could generate neoantigens, including missense mutations and inframe indels. A total 1,018 mutations (512 missense mutations from oncogenes, 488 missense mutations from tumor suppressors, 11 indels from oncogenes and 7 indels from tumor suppressors) were obtained (Marty et al., 2017, Cell, 171:1272-83). All mutations observed in TCGA patients that did not fall into the 200 most highly ranked cancer genes were designated passenger-like mutations. Furthermore, we created an additional set of established non-cancer mutations. To do so, we selected a set of genes that were known non-cancer genes and selected mutations in these genes regardless of their recurrence in TCGA (Table 1) (Lawrence et al., 2013, Nature, 499(7457):214-8).

TABLE 1
Set of known non-cancer genes.

| | | |
|---|---|---|
| OR2G6 | OR10G8 | OR2A5 |
| OR4C6 | OR5W2 | OR51S1 |
| OR4M2 | OR2T3 | OR9A2 |
| OR5L2 | OR10AG1 | OR51L1 |
| OR2T4 | OR4K1 | OR56A4 |
| OR5D18 | OR2M7 | OR52E2 |
| OR4A15 | OR4C12 | OR6M1 |
| OR6F1 | OR4D5 | OR2T11 |
| OR2T33 | OR2T1 | OR5M11 |
| OR4S2 | OR4P4 | OR4C46 |
| OR11L1 | OR5H14 | OR6K2 |
| OR4M1 | OR5F1 | OR2B3 |
| OR5T1 | OR2T8 | OR2T6 |
| OR8J3 | OR4C13 | OR56A1 |
| OR51B2 | OR5K1 | OR5B2 |
| OR8H2 | OR4K5 | OR4K15 |
| OR9G9 | OR2B11 | OR5AS1 |
| OR4N2 | OR5L1 | OR8A1 |
| OR10G9 | OR2L8 | OR4C3 |
| OR5I1 | ORCS1 | OR4D2 |
| OR14A16 | OR2T12 | OR8K3 |
| OR2M2 | OR2T34 | OR8J1 |
| OR5B12 | OR8H1 | OR4F6 |
| OR5M9 | OR5D16 | OR8H3 |
| OR4C11 | OR10Q1 | OR1J4 |
| OR1C1 | OR2M3 | OR52A5 |
| OR4N4 | OR6K3 | OR8B4 |
| OR5J2 | OR5T3 | OR51I1 |
| OR2G3 | OR14C36 | TTN |
| OR2T2 | ORCS3 | OR5H6 |
| OR4A16 | OR5AC2 | OR8I2 |
| OR52E6 | OR52J3 | OR5D14 |
| OR6N1 | OR4Q3 | OR8B2 |
| OR2AK2 | OR10A4 | OR4D11 |
| OR2L2 | OR4C16 | |

### Example 7—Selection of Other Classes of Residues

Peptides from pathogens, common germline human variants and randomly mutated human peptides were assembled for comparison with recurrent oncogenic mutations (Marty et al., 2017, Cell, 171:1272-83). The proteomes of 10 virus species and 10 bacterial species were downloaded from UniProt (UniProt Consortium, 2015). One thousand residues were selected at random from both the viral and the bacterial set. A random set of mutations was generated by sampling 3,000 possible amino acid substitutions across human proteins from Ensembl (release 90; GRCh38) (Aken et al., 2017, Nuc. Acids Res., 45(D1):D635-42). A set of 1,000 common germline variants was sampled from the Exome Variant Server.

### Example 8—Generating Mutant Peptide Sequences

To allow determination of peptide sequences incorporating missense mutations, protein sequences were obtained from Ensembl (release 90; GRCh38) (Aken et al., 2017, Nuc. Acids Res., 45(D1):D635-42) and updated with the new amino acid. For indels, we modified the corresponding mature messenger RNA transcript sequences (CDS) by inserting or deleting nucleotides, then translated the modified mRNA to protein sequence.

### Example 9—Patient Presentation Score-Based Clustering

A matrix of PHBR scores was constructed with 5,942 TCGA samples as rows, 1,018 recurrent oncogenic mutations as columns, and PHBR score in each cell. The matrix was clustered using hierarchical agglomerative clustering on rows and columns. For convenience of visualization, a partial matrix is displayed in FIG. 2. In order to use the dynamic range in heat map color to display variation in patient presentation scores relevant to MHC-II based presentation, the PHBR color scheme only varies from 0 to 40. Color bars provide additional information about patients and mutations, including ancestry, tumor type and T cell infiltration levels (patients) and mutation type and gene category (mutations). CD4 T cell infiltration was determined using CIBERSORT (Newman et al., 2015, Nat. Methods, 12(5):453-7), an mRNA-based immune infiltration prediction algorithm. Patients were mapped to high, medium-high, medium-low and low CD4+ T cell infiltration categories if their CIBERSORT scores fell into upper to lower quartiles respectively.

### Example 10—Comparison of Presentation Scores for Different Classes of Residue

PHBR presentation scores were calculated for 5,942 TCGA patients across different classes of residue including 71 highly-recurrent (>10) oncogenic missense mutations, 1000 random amino acid substitution, 1000 germline variants, 1000 viral residues and 1000 bacterial residues (see Selection of Other Classes of Residues). Across categories, this resulted in 24,189,882 PHBR scores (oncogenes: 231,738; tumor suppressor genes: 190,144; random: 5,942,000; common: 5,942,000; viral: 5,942,000; bacterial: 5,942,000). The distributions of PHBR scores in each category were compared with Mann-Whitney U tests and visualized with violin plots (FIG. 3A). Furthermore, we plotted cumulative distributions to demonstrate the practical presentation of each class across several thresholds and calculated the confidence intervals of each curve with bootstrapping (FIG. 3B; Table 1). Finally, we tested 20 independent sets of 1,000 random mutations to evaluate the confidence of the cumulative distributions (FIG. 10C).

### Example 11—Generation of Non-Cancer Population

As a control population, we used dbGaP samples (dbGaP: Phs000398, Phs000254, Phs000632, Phs000209, Phs000290, Phs000179, Phs000422, Phs000291, Phs000631 and Phs000518) typed at MHC-II using HLA-HD (Kawaguchi et al., 2017, Hum. Mutat. 38:788-97), with default parameters and typed at MHC-I using Optitype (Szolek et al., 2014, Bioinformatics, 30(23):3310-6), with default parameters. Both tools require germline (whole blood or tissue matched) whole exome sequenced samples. We successfully typed the HLA-I genes for 1,386 patients and the HLA-II genes for 1,219 patients who had alleles in the netMHCpan-3.0 and the netMHCIIpan-3.1 database. This control population was used to look at the MHC-II population of different classes of peptides by a non-cancer specific population (FIG. 10D). We would like to acknowledge the following dbGaP studies and all of their contributors:
Phs000398.v1.p1: The Atherosclerosis Risk in Communities Study is carried out as a collaborative study supported by National Heart, Lung, and Blood Institute contracts (HHSN268201100005C, HHSN268201100006C, HHSN268201100007C, HHSN268201100008C, HHSN268201100009C, HHSN268201100010C, HHSN268201100011C, and HHSN268201100012C). The authors thank the staff and participants of the ARIC study for their important contributions. This study is part of the NHLBI Grand Opportunity Exome Sequencing Project (GO-ESP). Funding for GO-ESP was provided by NHLBI grants RC2 HL103010 (HeartGO), RC2 HL102923 (LungGO) and RC2 HL102924 (WHISP). The exome sequencing was performed through NHLBI grants RC2 HL102925 (BroadGO) and RC2 HL102926 (SeattleGO). HeartGO gratefully acknowledges the following groups and individuals who provided biological samples or data for this study. DNA samples and phenotypic data were obtained from the following studies supported by the NHLBI: the Atherosclerosis Risk in Communities (ARIC) study, the Coronary Artery Risk Development in Young Adults (CARDIA) study, Cardiovascular Health Study (CHS), the Framingham Heart Study (FHS), the Jackson Heart Study (JHS) and the Multi-Ethnic Study of Atherosclerosis (MESA).
Phs000254.v2.p1: This study is part of the NHLBI Grand Opportunity Exome Sequencing Project (GO-ESP). Funding for GO-ESP was provided by NHLBI grants RC2 HL103010 (HeartGO), RC2 HL102923 (LungGO) and RC2 HL102924 (WHISP). The exome sequencing was performed through NHLBI grants RC2 HL102925 (BroadGO) and RC2 HL102926 (SeattleGO). Collection of the cystic fibrosis data and specimens was supported by Awards GIBSONO7K0, KNOWLE00A0, OBSERV04K0, and RDP R026 from the Cystic Fibrosis Foundation; NHLBI grants R01 HL068890 and R01 HL095396; NCRR grant UL1RR025014 and NHGRI grant R00 HG004316.
Phs000632.v1.p1: This study is part of the NHLBI Grand Opportunity Exome Sequencing Project (GO-ESP). Funding for GO-ESP was provided by NHLBI grants RC2 HL103010 (HeartGO), RC2 HL102923 (LungGO) and RC2 HL102924 (WHISP). The exome sequencing was performed through NHLBI grants RC2 HL102925 (BroadGO) and RC2 HL102926 (SeattleGO). The Hematological Cancer specimens and data were collected in the laboratory of Dr. Benjamin L. Ebert, Brigham & Womens Hospital/Broad Institute, Boston, USA.
Phs000209.v13.p3: MESA and the MESA SHARe project are conducted and supported by the National Heart, Lung, and Blood Institute (NHLBI) in collaboration with MESA investigators. Support for MESA is provided by contracts N01-HC95159, N01-HC-95160, N01-HC-95161, N01-HC-95162, N01-HC-95163, N01-HC-95164, N01-HC-95165, N01-HC95166, N01-HC-95167, N01-HC-95168, N01-HC-95169, UL1-RR-025005, and UL1-TR-000040.
Phs000290.v1.p1: Exome data provided by ARRA-NHLBI Lung Cohorts Sequencing Project 1RC2HL102923-01. The authors wish to thank the supported effort of the faculty and staff members of the Johns Hopkins University Bayview Genetics Research Facility and the Johns Hopkins University ‘Genomics and Genetics of Pulmonary Arterial Hypertension’ program (NIH P50 HL084946, P. M. Hassoun, NIH K23 AR52742-01, L. K. Hummers, and NHLBI F32 HL083714-01 S. C. Mathai).
Phs000179.v5.p2: This research used data generated by the COPDGene study, which was supported by NIH grants U01HL089856 and U01HL089897. The COPDGene project is also supported by the COPD Foundation through contributions made by an Industry Advisory Board comprised of Pfizer, AstraZeneca, Boehringer Ingelheim, Novartis, and Sunovion.
Phs000422.v1.p1: This study is part of the NHLBI Grand Opportunity Exome Sequencing Project (GO-ESP). Funding for GO-ESP was provided by NHLBI grants RC2 HL103010 (HeartGO), RC2 HL102923 (LungGO) and RC2 HL102924 (WHISP). The exome sequencing was performed through NHLBI grants RC2 HL102925 (BroadGO) and RC2 HL102926 (SeattleGO). The following NHLBI Severe Asthma Research Program (SARP) sites have contributed parent study data and DNA samples for exome sequencing in this project: Wake Forest School of Medicine (R01 HL069167), University of Wisconsin (R01 HL069116), University of Virginia, Cleveland Clinic (R01 HL069170), National Jewish Health, University of Pittsburgh (R01 HL069174), Washington University (R01 HL069149), Brigham and Women's Hospital (R01 HL069349) and genotyping was supported by NHLBI HL87665 and 1RC2 HL101487).
Phs000291.v2.p1: This study is part of the NHLBI Grand Opportunity Exome Sequencing Project (GOESP). Funding for GO-ESP was provided by NHLBI grants RC2 HL103010 (HeartGO), RC2 HL102923 (LungGO) and RC2 HL102924 (WHISP). The exome sequencing was performed through NHLBI grants RC2 HL102925 (BroadGO) and RC2 HL102926 (SeattleGO). The authors wish to thank the supported effort of the faculty and staff members of the Johns Hopkins University Bayview Genetics Research Facility, NHLBI grant HL066583 (Garcia/Barnes, PI) and NHGRI grant HG004738 (Barnes/Hansel, PI). The Lung Health Study was supported by U.S. Government Contract No. N01-HR-46002 from the Division of Lung Diseases of the National Heart, Lung and Blood Institute. The principal investigators and senior staff of the clinical and coordinating centers, the NHLBI, and members of the Safety and Data Monitoring Board of the Lung Health Study can be found at biostat.umn.edu/lhs/ on the World Wide Web and as follows: Case Western Reserve University, Cleveland, Ohio: M. D. Altose, M.D. (Principal Investigator), C. D. Deitz, Ph.D. (Project Coordinator); Henry Ford Hospital, Detroit, Mich.: M. S. Eichenhorn, M.D. (Principal Investigator), K. J. Braden, A. A. S. (Project Coordinator), R. L. Jentons, M.A.L.L.P. (Project Coordinator); Johns Hopkins University School of Medicine, Baltimore, Md.: R. A. Wise, M.D. (Principal Investigator), C. S. Rand, Ph.D. (Co-Principal Investigator), K. A. Schiller (Project Coordinator); Mayo Clinic, Rochester, Minn.: P. D. Scanlon, M.D. (Principal Investigator), G. M. Caron (Project Coordinator), K. S. Mieras, L. C. Walters; Oregon Health Sciences University, Portland: A. S. Buist, M.D. (Principal Investigator), L. R. Johnson, Ph.D. (LHS Pulmonary Function Coordinator), V. J. Bortz (Project Coordinator); University of Alabama at Birmingham: W. C. Bailey, M.D. (Principal Investigator), L. B. Gerald, Ph.D., M. S.P.H. (Project Coordinator); University of California, Los Angeles: D. P. Tashkin, M.D. (Principal Investigator), I. P. Zuniga (Project Coordinator); University of Manitoba, Winnipeg: N. R. Anthonisen, M.D. (Principal Investigator, Steering Committee Chair), J. Manfreda, M.D. (Co-Principal Investigator), R. P. Murray, Ph.D. (Co-Principal Investigator), S. C. Rempel-Rossum (Project Coordinator); University of Minnesota Coordinating Center, Minneapolis: J. E. Connett, Ph.D. (Principal Investigator), P. L. Enright, M.D., P.G. Genomics & Genetics of the Lung Health Study Jun. 10, 2011 version Page 6 of 8 Lindgren, M. S., P. O'Hara, Ph.D., (LHS Intervention Coordinator), M. A. Skeans, M. S., H. T. Voelker; University of Pittsburgh, Pittsburgh, Pa.: R. M. Rogers, M.D. (Principal Investigator), M. E. Pusateri (Project Coordinator); University of Utah, Salt Lake City: R. E. Kanner, M.D. (Principal Investigator), G. M. Villegas (Project Coordinator); Safety and Data Monitoring Board: M. Becklake, M.D., B. Burrows, M.D. (deceased), P. Cleary, Ph.D., P. Kimbel, M.D. (Chairperson; deceased), L. Nett, R. N., R. R. T. (former member), J. K. Ockene, Ph.D., R. M. Senior, M.D. (Chairperson), G. L. Snider, M.D., W. Spitzer, M.D. (former member), O.D. Williams, Ph.D.; Morbidity and Mortality Review Board: T. E. Cuddy, M.D., R. S. Fontana, M.D., R. E. Hyatt, M.D., C. T. Lambrew, M.D., B. A. Mason, M.D., D. M. Mintzer, M.D., R. B. Wray, M.D.; National Heart, Lung, and Blood Institute staff, Bethesda, Md.: S. S. Hurd, Ph.D. (Former Director, Division of Lung Diseases), J. P. Kiley, Ph.D. (Former Project Officer and Director, Division of Lung Diseases), G. Weinmann, M.D. (Former Project Officer and Director, Airway Biology and Disease Program, DLD), M. C. Wu, Ph.D. (Division of Cardiovascular Sciences).
Phs000631.v1.p1: The datasets were obtained as part of the identification of SNPs Predisposing to Altered ALI Risk (iSPAAR) study funded by the NHLBI (RC2 HL101779).
Phs000518.v1.p1: The authors wish to acknowledge the support of the National Heart, Lung and Blood Institute (NHLBI) and the contributions of the research institutions, study investigators, field staff and study participants in creating this resource for biomedical research. This work was supported in part by grants R01 HL071798 from the NHLBI and U54 HL096458 from the NHLBI (previously supported by the NCRR), the components of NIH. This study is part of the NHLBI Grand Opportunity Exome Sequencing Project (GO-ESP). Funding for GO-ESP was provided by NHLBI grants RC2 HL103010 (HeartGO), RC2 HL102923 (LungGO) and RC2 HL102924 (WHISP). The exome sequencing was performed through NHLBI grants RC2 HL102925 (BroadGO) and RC2 HL102926 (SeattleGO).

### Example 12—Analysis of Presentation Versus Mutation Frequency Among Tumors

The PHBR scores of 5,942 patients in TCGA were calculated for 1000 passenger mutations (observed 1 or 2 times in the 5,942 patients; not occurring in 200 cancer-implicated genes). PHBR scores were calculated for 1,018 recurrent driver mutations (from 200 cancer implicated genes) in the 7137 patients. The distribution of passenger PHBR scores was compared to 841 low frequency (≤5 times), 149 medium frequency (>5, ≤20 times) and 28 high frequency oncogenic mutations (>20 times). The distributions of PHBR scores in each category were compared with Mann-Whitney U tests and visualized with violin plots (FIG. 3C). Furthermore, we plotted cumulative distributions to demonstrate the practical presentation of each frequency grouping across several thresholds (FIG. 3D).

### Example 13—Modeling the Effect of PHBR-II on Mutation Probability

To assess the role of MHC-II in regards to mutation probability, we further restricted the recurrent oncogenic mutations to those occurring at least two times in the set of patients, resulting in 787 mutations and 5,942 patients. To first visualize the difference in PHBR-II distributions for mutations observed versus absent from tumors, PHBR-II scores from the 1,018 mutations×5,942 patient matrix were grouped according to mutation status and plotted in side-by-side violin plots. Next, we built a 5,942×787 binary mutation matrix yᵢⱼ ∈{0, 1} indicating whether patient i has a specific mutation j. We evaluated the relationship between this binary matrix and the matched 5,942×787 matrix with PHBR-II scores xᵢⱼ of patient i and for mutation j. We fitted a generalized additive model for the PHBR-II score and mutation probability with the GAM function in the MGCV R package (Wood, 2001, R. News, 1:20-5). To estimate the effect of xᵢⱼ on yij, we considered the following random effects model:

logit(P(yᵢⱼ=1|xᵢⱼ))=ηi+γ log(xᵢⱼ)

where ηᵢ˜N(0, θ_{η}) are random effects capturing different mutation propensities among patients.

In these models, γ measures the effect of the log-PHBR-II. We fitted this model using the glmer function from the lme4 R package (Bates et al., 2015, J. Stat. Softw. 67:1-48) and tested the null hypothesis that γ=0. To analyze the PHBR-mutation relationship in different tumor types, we fit separate models for each tumor type where there were at least 50 total number of driver mutations in the cohort. Furthermore, we used this same method to evaluate the difference in selection between mutations high allelic fraction and low allelic fraction (see ‘Clonality of mutations’ section).

### Example 14—Modeling the Interaction Between MHC-I and MHC-II Effects

To assess the interaction between MHC-I and MHC-II in regards to mutation probability, we reduced the set of patients to those successfully typed for both MHC-I and MHC-II (Marty et al., 2017, Cell, 171:1272-83). We further restricted the recurrent oncogenic mutations to those occurring at least twice in the set of patients, resulting in 787 mutations and 5,942 patients. Then, we checked the correlation between MHC-I and MHC-II presentation using a Spearman Rank Test between MHC-I and MHC-II scores for each patient across all 1,018 mutations. These correlations were displayed as a histogram (FIG. 10B). After finding low correlation scores, we built a model of the interaction.

We built a 5,942×787 binary mutation matrix yᵢⱼ ∈{0, 1} indicating whether patient i has a specific mutation j. We evaluated the relationship between this binary matrix and two matched 5,942×787 matrices with MHC-I PHBR scores wᵢⱼ of patient i and for mutation j and MHC-II PHBR scores xᵢⱼ of patient i and for mutation j. To visualize the relationship between wᵢⱼ and xᵢⱼ with yᵢⱼ, we fit an generalized additive model for the PHBR scores of both classes using the GAM function in the mgcv R package (Wood, 2001, R. News, 1:20-5). Finally, to estimate the effect of xᵢⱼ and wᵢⱼ on _{yij}, we considered the following random effects model:

A within-patient model relating xᵢⱼ and wᵢⱼ to yᵢⱼ for a given patient

logit(P(yᵢⱼ=1|xᵢⱼ,wᵢⱼ))=α+ηᵢ+γ log(xᵢⱼ)+β log(wᵢⱼ)

where α is the intercept term and ηᵢ˜N(0, θ_{η}) are random effects capturing different mutation propensities among patients.

In these models, γ measures the effect of the log-PHBR-I and β measures the effect of the log-PHBR-II on the probability of a mutation being observed. We fitted this model using the glmer function from the lme4 R package (Bates et al., 2015, J. Stat. Softw. 67:1-48) and tested the null hypothesis that γ=0 and β=0. To analyze the PHBR-mutation relationship in different tumor types, we fit separate models for each tumor type where there were at least 50 total number of driver mutations in the cohort. Given the distinct PHBR score ranges for MHC-I and MHC-II, we constructed an OR analysis to compare the relative effects in the population. Instead of reporting the OR for a single unit increase, we reported the odds of observing a mutation in the 25th PHBR percentile relative to the 75th PHBR percentile.

### Example 15—Fraction of Patients with Presentation

For each mutation in our set of 1,018 driver mutations, we calculated the fraction of patients that could present the mutation based on their MHC-I and MHC-II genotype, respectively. We used the standard weak binding cutoffs of 2 for MHC-I and 10 for MHC-II. These results were visualized with a density plot (FIG. 5D) and a scatterplot of the high frequency mutations (FIG. 11D). Furthermore, we compared the distributions for fraction of MHC-I and MHC-II presentation across several thresholds (0.25, 0.5, 1, and 2 for MHC-I and 1, 2, 5, and 10 for MHC-II) to ensure robustness (FIG. 11E).

### Example 16—Clonality of Mutations

The occurrences of mutations within the set of 1,018 driver mutations were designated as likely clonal or likely subclonal based on the allelic fraction annotation provided by TCGA. Mutations that were among the lowest 30th percentile were designated likely subclonal and all the remaining were considered likely clonal. We modeled the independent effect of PHBR-II and PHBR-I on mutation probability separately for subclonal and clonal occurrences as described above in the section ‘Modeling the effect of PHBR-II on mutation probability’.

### Example 17—MHC-Based Selection with Different Immune Infiltration Phenotypes

Immune infiltration levels were quantified from expression using CIBERSORT

(Newman et al., 2015, Nat. Methods, 12(5):453-7) and patient-specific cytotoxicity scores were derived (Rooney et al., 2015, Cell, 160:48-61). Tumors were divided into “high” and “low” groups for each of the following categories using the tumor-type specific 30th and 70th percentile: APC infiltration (B cells, dendritic cells and macrophages), cytolytic activity, CD8+ T cell infiltration and CD4+ T cell infiltration. We modeled the independent effect of PHBR-II and PHBR-I on mutation probability in the high and low groups as described above in the section ‘Modeling the effect of PHBR-II on mutation probability’.

### Example 18—MHC Coverage

MHC-I and MHC-II coverage of driver mutations was determined by calculating the fraction of the 1,018 driver mutation PHBR scores for each patient that fell below the binding thresholds, 2 and 10 for MHC-I and MHC-II respectively. This analysis resulted in each patient being assigned two MHC coverage values (MHC-I and MHC-II). Furthermore, two more values were calculated for each patient using 1,000 passenger mutations. The number of homozygous genes was determined for each patient by adding the number of identical alleles for MHC-I (-A, -B, -C) and MHC-II (-DRB, -DPA, -DPB, -DQA, -DQB) separately. The MHC coverage values were calculated for these patients as well and compared to the TCGA MHC coverage values with a Mann Whitney U test.

### Example 19—Age at Diagnosis Analysis

To visualize the association between MHC coverage and age at diagnosis, the patients with MHC coverage values in the lowest quartile and the patients with MHC coverage values in the highest quartile were compared. To determine statistical significance, a linear model in R was applied with age as the independent variable and MHC coverage, ancestry and tumor type as the dependent variables. Statistical significance was also determined for MHC-I and MHC-II coverage of passenger mutations and MHC homozygosity count as a replacement for MHC coverage. To assess the practical effect size of the extreme cases of MHC coverage, we compared the ages at diagnosis of the 5% of patients with the lowest MHC-I coverage with the ages at diagnosis for the 5% of patients with the highest MHC-I coverage with a two sample t test. We also performed the same analysis for the patients with the highest and lowest 10% of MHC-I coverage. A Pearson correlation test was used to determine the correlation between MHC coverage of driver mutations and MHC coverage of passenger mutations for both MHC-I and MHC-II.

### Example 20—Quantification and Statistical Analysis

For all individual tests, a p value of less than 0.05 was considered significant. When multiple comparisons were made, p values were adjusted using the Benjamini-Hochberg method unless otherwise specified. For all box plots, whiskers indicate the 1.5 IQR range.

The python (2.7) and R code used to perform the analyses described in this manuscript and generate all main and supplemental figures is available in Data 51 and at github.com/Rachelmarty20/MHC_II on the World Wide Web.

### Example 21—Creating an Affinity-Based MHC-II Genotype Scoring Scheme

To study the role of MHC-II during tumorigenesis, we needed a score linking MHC-II genotype to presentation of specific mutations. We first constructed a score representing the ability of a single MHC-II molecule to present a residue. We previously established that using the best rank among peptides provided the best performance for predicting MHC-I presentation. We therefore adapted this scoring scheme to reflect the structure and composition of MHC-II. Three molecules (HLA-DR, HLA-DP, and HLA-DQ) make up the MHC-II, all of which are heterodimers formed by an alpha and beta chain. Both the alpha and the beta chain influence the binding affinity of a peptide. In contrast to MHC-I, the MHC-II binding groove is open at both ends, allowing longer peptides to bind. To predict binding affinity to each alpha- and beta-paired MHC-II molecule, we used netMHCIIpan-3.1 that returns a single rank for the pair with each peptide (Karosiene et al., 2013, Immunogenetics, 65:711-24). Unlike netMHCpan-3.0, netMHCIIpan-3.1 has only been optimized for 15-mers and not for varying lengths. As with MHC-I, we assigned the single MHC-II molecule presentation score as the best rank of all k-mers containing the desired residue (FIG. 1A).

Next, single molecule residue-centric presentation scores were combined into an MHC-II genotype score. Previously, MHC-I single allele best rank scores were combined using the harmonic mean resulting in the patient best-rank harmonic mean (PHBR-I) score, as this outperformed all other tested formulations. To create an analogous score for MHC-II, we modified the PHBR-I score to account for the different composition of MHC-II molecules. The MHC-II genotype comprises two copies each of HLADR alpha and beta, HLA-DP alpha and beta and HLA-DR alpha and beta. HLA-DRA is the only non-variable gene in the population, resulting in only two possible HLA-DR heterodimers. Each individual can form four possible alpha-beta heterodimers from HLA-DP and HLA-DQ. This results in a total of ten possible unique heterodimeric MHC-II molecules (FIG. 1B). To weight each gene equally in the final presentation score, each HLA-DRB1 allele is considered twice, bringing the total number of complexes to twelve. To evaluate the combined effect of these complexes on the presentation of a residue, the best rank score is calculated for all twelve complexes and those twelve values are combined using the harmonic mean to create a PHBR-II score (FIG. 1C).

To assess the performance of the PHBR-II score at predicting extracellular presentation, we compared the scores for peptides derived from several multi-allelic HLA-DR expressing cell lines against matched scores for randomly derived peptides (Ciudad et al., 2017, J. Leukoc. Biol., 101:15-27) (FIG. 1D). The combined AUC across all cell lines was 0.69 (FIG. 1E). This formulation of the PHBR-II score outperformed another scoring variation where peptides of varying lengths were considered (FIG. 7). Two reasons contribute to the reduced performance relative to MHC-I (receiver operating characteristic curve [ROC] area under the curve [AUC] 0.75) (Marty et al., 2017, Cell, 171:1272-83). First, predicting single allele MHC-II binding has higher error than predicting single allele MHC-I binding. Second, computing an AUC value requires a non-binding negative set of residues. We employ a random set of residues when evaluating PHBR scores for both MHC classes; however, MHC-II has a larger effective binding range than MHC-I. As a result, the negative set should have an order of magnitude more actual binding residues for MHC-II than MHC-I. Thus, lack of an appropriate negative set for MHC-II deflates the calculated AUC value. For this application, namely using predicted MHC class II binding affinities to identify T cell epitopes for which the exact restricting MHC class II molecule is not known, performance measured by AUC values is typically around 0.7. Despite these limitations, the PHBR-II score contains significant signal that renders it useful for further analysis.

Finally, we applied the HLA-HD tool (Kawaguchi et al., 2017, Hum. Mutat. 38:788-97) to predict HLA-II alleles for patients in TCGA with exome sequencing data (see Table S1 in doi.org/10.1016/j.cell.2018.08.048 on the World Wide Web). To the best of our knowledge, HLA-HD is currently the only tool that can call alpha and beta alleles for HLA-DR, HLA-DP, and HLA-DQ with high accuracy. Thus, from a total of 8,333 patients with exome sequencing, we successfully typed 7,929 patients at all three genes. To validate these HLA types, we also applied xHLA (Xie et al., 2017, PNAS USA, 114: 8059-64), which calls the beta alleles for HLA-DR, HLA-DP, and HLA-DQ. We restricted our patient set to samples where both HLA-HD and xHLA completely agreed, leaving 5,942 patients (FIG. 8A; see Table S1 in doi.org/10.1016/j.cell.2018.08.048 on the World Wide Web). Within the typed TCGA patients, HLA-DPA1 revealed the least population variation, with only 14 types represented and the most common allele (HLA-DPA1*0103) at a frequency of 0.76 in the population. HLA-DRB1 had the most variation in the population, with 74 types represented, the most common of which (HLA-DRB1*0701) was observed at only a frequency of 0.20 (FIGS. 8B-8F).

### Example 22—Recurrent Cancer Mutations are Poorly Presented by Human MHC-II

Mutations that drive the early development of tumors should be observed more frequently across tumors. We therefore used recurrence of mutations in established oncogenes and tumor suppressors as criteria to assemble a list of 1,018 cancer-driving mutations likely to have occurred prior to immune evasion and that could therefore reflect the effects of selection by immunosurveillance. We calculated PHBR-II scores for every mutation-patient combination, resulting in a matrix of 5,942 patients (FIG. 2, rows; see Table S2 in doi.org/10.1016/j.cell.2018.08.048 on the World Wide Web) and 1,018 mutations (FIG. 2, columns). The matrix provides a high level overview of the MHC-II presentation landscape across cancer patients and recurrent cancer mutations. Patients and mutations were clustered according to similarity of presentation score profiles. While we observed no obvious clustering of patients by tumor type or infiltration by CD4+ T cells, we did observe expected clusters of samples with shared ancestry, resulting from population-specific differences in MHC-II allele frequencies. Interestingly, we observed bias toward poor presentation of tumor suppressor mutations by MHC-II across the entire population (Fisher's exact test, PHBR-II R10, OR [odds ratio]=1.43, p=0.006). Notably, this same enrichment was not present for MHC-I presentation (Fisher's exact test, PHBR-I R2, OR=1.33, p=0.40). Although only a small fraction of the tested mutations were in-frame indels, there was no clear difference between the MHC-II presentation of missense mutations and indels. Interestingly, when a similar matrix was generated using the wild-type sequences instead of the mutations, the presentation of the sequences across the population were highly concordant (Pearson's r=0.96, FIGS. 9A and 9B).

Next, we compared the ability of the 5,942 cancer patients to present different classes of residues by MHC-II. We calculated the PHBR-II scores of every patient for 1,000 viral residues, 1,000 bacterial residues, 1,000 common polymorphisms, and 1,000 random mutations (Marty et al., 2017, Cell, 171:1272-83). To compare the behaviors of PHBR-II scores, we visualized raw distribution and the cumulative distribution function (CDF) for each class of residues. Viral and bacterial residues were presented the most effectively out of these classes by the patients in the population (FIG. 3A). Assuming that the MHC-II system has primarily evolved to ward off pathogens, it is not surprising that the CDF curves are shifted to the left in comparison with other classes, with more than 27% of viral and 29% of bacterial PHBR-II scores falling below a PHBR-II threshold of 6 (threshold based on 0.2 false-positive rate) (FIGS. 3B and 10A; Table 2 for confidence intervals [CI]). Common germline polymorphisms and random mutations should, in contrast, approximate events that are selectively neutral. MHC-II presentation of germline variants should in principle be decoupled by tolerance such that germline variants should not be biased to occur in particularly well or poorly presented peptides. Similarly, randomly selected mutations should represent an unbiased sample of background MHC-II presentation. Consistent with positive selection, pathogen residues are presented significantly better than germline variants or random mutations by MHC-II across the population, yet 22% and 23% of PHBR-II scores still fall below the 6 PHBR-II threshold for common germline polymorphisms and random mutations, respectively. In contrast, distributions of PHBR-II scores for recurrent mutations in oncogenes and tumor suppressors (observed >10 times in MHC-II-typed population) show a shift upward toward poor presentation relative to random mutations (p<2.2e±16), with only 12% of scores for mutations in oncogenes falling below the 6 PHBR-II threshold. Strikingly, there was even poorer presentation of mutations in tumor suppressor genes (p<2.2e±16; relative to random mutations), with only 7% of PHBR-II scores below the 6 PHBR-II threshold. The differences observed in MHC-II presentation for these classes of mutation were robust to the inclusion of less recurrent (observed >2 times in TCGA) cancer mutations (FIG. 10B) and to using different samples of random mutations (FIG. 10C, empirical p<0.05). Interestingly, these trends were not unique to cancer patients but were also observed in alternate human populations, suggesting that MHC-II genotypes do not significantly differ between the two populations (FIG. 10D).

TABLE 2
Fraction of residues with MHC-II presentation in different
peptide classes.

| | Fraction | 95% CI |
|---|---|---|
| Oncogenes | 0.120 | (0.119, 0.121) |
| Tumor suppressor genes | 0.0649 | (0.0641, 0.0657) |
| Random | 0.236 | (0.236, 0.236) |
| Germline | 0.222 | (0.222, 0.222) |
| Viral | 0.272 | (0.272, 0.273) |
| Bacterial | 0.286 | (0.286, 0.287) |

We next evaluated whether the recurrence of a mutation was related to its presentation by MHC-II by comparing the PHBR-II score distributions of passenger mutations and varying frequencies of cancer-driving mutations (FIG. 3C). Passenger mutations, defined as mutations occurring only 1-2 times across all tumors in non-cancer genes, had a PHBR-II score distribution very similar to that of random mutations with an enrichment for PHBR-II scores near 0, suggesting that many passengers are likely to be effectively presented. This enrichment of presented passenger mutations is consistent with recent reports that HLA loss of heterozygosity is frequent in some tumor types and is associated with the accumulation of mutations that would have been effectively presented by the lost allele. Consequently, 25% of the passenger mutation PHBR-II scores fall below the PHBR-II cutoff of 6 (FIG. 3D). In comparison, we observed significantly worse presentation with increasing mutation frequency for recurrent mutations (observed >2 times across typed tumors) in known cancer genes (p<2.2e±14). The percentage of PHBR-II scores falling below the PHBR-II threshold of 6 falls with each jump in frequency; from 20% for low frequency driver mutations (≤5 times; 841 total) to 16% for medium frequency driver mutations (>5, ≤20 times; 149 total) to a dramatic 8% for high frequency driver mutations (>20 times; 28 total) (FIG. 3D). Despite the striking shift toward larger PHBR-II scores with increasing recurrence, MHC-II presentation across patients was not quite significantly correlated with mutation frequency (burden) across tumors overall (Spearman's rho=0.27, p=0.07, FIG. 10E). This is in contrast to the relationship observed for MHC-I (Spearman's rho=0.66, p=1.02e±6 within the same patient group). We note that median PHBR-II scores for mutations observed >10 times tend to be elevated equivalently. This may reflect a threshold beyond which presentation no longer occurs and thus beyond which numeric differences in PHBR-II score should no longer be informative about mutation frequency. Taken together, these results suggest that MHC-II-based presentation across the human population constrains the frequency at which mutations arise across tumors.

### Example 23—MHC-II Genotype Constrains the Landscape of Cancer Mutations in Individual Tumors

Given observed bias for cancer mutations to be poorly presented by human MHC-II (FIG. 3A), we hypothesized that MHC-II genotype could influence patient-specific mutation probability. To explore this hypothesis, we intersected occurrence of mutations with potential of an individual to present those mutations as quantified by their PHBR-II score. PHBR-II scores were separated into two groups: those that corresponded to observed mutations and those that corresponded to unobserved mutations (FIG. 4A). Consistent with our hypothesis, we observed a large upward shift in PHBR-II distribution for the observed mutations as opposed to the unobserved mutations. As mutations become less presentable (higher PHBR-II), the probability of mutation increases significantly (FIG. 4B), with the most pronounced increase occurring at lower PHBR-II scores.

Next, we used a logistic regression with non-linear effects to model the relationship between MHC-II genotype and the probability of observing a recurrent somatic mutation in a pan-cancer setting. We found a substantial increase in odds of acquiring a mutation as PHBR-II scores increased (OR=1.23, p<9.9e±58, Table 3). Importantly, passenger mutations, established non-driver mutations (Table 1), and germline polymorphisms did not exhibit the same increase (OR=1.00, OR=0.99, and OR=0.99, respectively, Table 3). In addition, the OR decreased when less stringent HLA type calls were used (OR=1.20), suggesting the importance of accurate HLA typing.

TABLE 3
The association between PHBR-II score and mutation occurrence

| | MHC-II PHBR | | |
|---|---|---|---|
| | OR | 95% Cl | p Value |
| ≥2 mutation | 1.23 | (1.19, 1.26) | 9.9e−58 |
| Passenger mutations | 1.00 | (0.94, 1.06) | 0.99 |
| Non-driver mutations | 0.99 | (0.06, 1.04) | 0.96 |
| Germline variants | 0.99 | (0.99, 0.99) | 5.8e−07 |

OR, 95% Cl, and p value are shown for logistic regression model relating PHBR-II scores to set of mutations observed ≥2 times in set of tumors. Models relating PHBR-II score to sets of passenger mutations, non-driver mutations, and germline variants serve as controls.
CI, confidence interval; OR, odds ratio.

Because the immune environment can vary considerably across tissue sites, we revisited our analysis for each tumor type separately (FIG. 4C; see Table S5 at doi.org/10.1016/j.cell.2018.08.048 on the World Wide Web). Twelve of the eighteen tissues had significant positive ORs (p<0.05) after multiple testing correction. Similar to MHC-I, MHC-II genotype had the strongest effect in thyroid cancer; however, the effects of MHC-II were even greater than MHC-I (OR=2.63 versus OR=2.21, considering only thyroid cancer patients with confident MHC-I and MHC-II typing) (FIG. 4C).

### Example 24—MHC-II Works Together with MHC-I to Influence Mutation Probability in Individual Tumors

We previously established the influence of germline MHC-I genotype on the probability of observing specific mutations in tumors (Marty et al., 2017, Cell, 171:1272-83). To assess the combined influence of MHC-I and MHC-II on mutation probability, we evaluated the correlation between PHBR-I and -II scores across recurrent cancer mutations. The range and distribution of PHBR-I and -II scores differs substantially (FIG. 11A), and while lower PHBR scores are indicative of more effective presentation in both cases, the range of values where most presentation takes place is expected to differ as MHC-II binds peptides with lesser stringency for peptide affinity and more promiscuity than MHC-I. These differences suggest the potential for MHC-I and MHC-II to contribute to presentation and, thus, constrain mutation probability in complementary ways. Indeed, we observed only a weak positive correlation between PHBR-I and -II score distributions across recurrent cancer mutations (Spearman's rho=0.36; FIGS. 5A and 11B). Consequently, we modeled the relationship between the probability of observing a mutation and both classes of PHBR scores across the 1,018 recurrent mutations (FIG. 5B). Mutations with low PHBR scores (effective presentation) for either class had a much lower probability of being observed in tumors than mutations that had high PHBR scores (poor presentation) for both classes.

To quantify the influence of MHC-I and MHC-II on probability of mutation, we used an additive logistic regression model with non-linear effects that incorporated both PHBR-I and -II scores in the pan-cancer setting. Because the distributions of PHBR-I and -II are very different, we calculated the ORs between the 25th and 75th percentile PHBR, such that the OR represents the increase in odds of observing a mutation among individuals with a high PHBR score relative to a low PHBR score for each MHC class. Notably, we found the impact of MHC-II on the probability of a mutation to be larger than the impact of MHC-I (single model incorporating both classes: OR=1.74 with CI [1.67, 1.80] and OR=1.60 with CI [1.54, 1.64], respectively). To better understand the relative effects of presentation by MHC II versus MHC I in a tissue-specific setting, we also estimated their individual effects on mutation probability in a joint model. Consistent with our pan-cancer analysis, we found MHC-II to have more extreme effect sizes in most tissues (FIG. 11C).

The same driver mutations can occur early or late during tumor development; however, in a model where immune selection is impaired later in tumorigenesis by mechanisms of immune evasion, selection should be stronger on early clonal occurrences. Therefore, we further annotated mutations according to whether they were more likely clonal or subclonal based on relative allelic fraction of the mutations (STAR Methods). Consistent with our assumption, likely subclonal mutations had decreased ORs relative to PHBR II and PHBR I scores (single class model, reference Table 3: PHBR-II OR=1.13 as compared to 1.21 for all mutations, PHBR-I OR=1.16 as compared to 1.20 for all mutations, FIG. 5C), confirming that subclonal events are subject to weaker selection. Moreover, when restricting analysis of selection to likely clonal mutations, ORs for both PHBR II and PBHR I scores increased (single class model, reference Table 1: PHBR-II OR=1.29 as compared to 1.21 for all mutations, PHBR-I OR=1.29 as compared to 1.20 for all mutations). Although mutation calls may be less confident for subclonal mutations, these results suggest that true effect sizes may be higher than previously reported.

### Example 25—Differences in MHC-II Versus MHC-I Presentation Specificities

Next, we explored whether practical differences exist in the presentation of particular driver mutations by MHC-II versus MHC-I. We compared the fraction of patients wherein a mutation was presented by MHC-II with the same fraction for MHC-I (FIG. 5D; Appendix A) and further divided mutations into four categories: rarely presented by either MHC-I or MHC-II, more frequently presented by MHC-I, more frequently presented by MHC-II, and frequently presented by both. Interestingly, we observed that MHC-II-based presentation tended to be bimodal, such that a mutation was presented by most patients, or by almost no patients, with a few notable exceptions including KRAS G12 (FIG. 11D). In contrast, MHC-I-based presentation spanned the full range, with many mutations presented in varying fractions of patients. Although these trends may be impacted by the higher sensitivity of the PHBR-I score as compared to the PHBR-II score, they were constant across several thresholds (FIG. 11E). This suggests that MHC-II-based presentation may be more shared across patients, whereas MHC-I-based presentation is more individual-specific. We further investigated the mutations frequently presented by both MHC-I and MHC-II, because we would expect them to arise with low likelihood in cancer. Indeed, these mutations had lower allelic fractions than mutations presented well by at least MHC-I or MHC-II (Mann-Whitney, p=0.03), suggesting these mutations are subclonal, arising after immune evasion, and could be effectively eliminated by the immune system.

Based on this analysis, the relative abundance of class I peptides appears to be higher than that for class II, suggesting better potential for engineering class I anti-tumor responses; however, recent reports suggest a bias for responses to be CD4+-driven in practice. This could indicate that TCR availability is a major bottleneck for effective CD8+ immune responses.

### Example 26—Evidence for Distinct Effects of Class II- Versus Class I-Driven Immunosurveillance

Differences in the dynamics of peptide presentation and immune response for MHC-I versus MHC-II may have important implications for tumor-immune interactions. Whereas MHC-I binds peptides with high specificity, MHC-II binds a broader array of peptides with a high degree of promiscuity. CD4+ T cells activated by MHC-II-peptide complexes can play either a regulatory or an effector role, whereas CD8+ T cells are strictly (cytotoxic) effectors. The different properties of class I- and class II-based immunity are essential for an effective defense against pathogens, but the implications for anti-tumor responses are less clear. We therefore sought to further quantify the potential for these distinct roles to introduce measurable differences between class I- and class II-mediated immunosurveillance during tumor development. Because of its established regulatory role in cancer, we reasoned that MHC II-driven immunosurveillance could have a larger effect on the immune microenvironment than MHCI. Using CIBERSORT (Newman et al., 2015, Nat. Methods, 12(5):453-7) to evaluate infiltration by different immune cell types into tumors, we sought to identify a relationship between immune infiltrates, cytotoxicity score (Rooney et al., 2015, Cell, 160:48-61), and strength of immune selection. We divided patients into groups based on their immune infiltrates and cytotoxicity scores and tested for differences in immune selection (FIGS. 12A-12D) but did not find any significant relationships. This apparent lack could be an artifact of the timing of the MHC-imposed selection relative to when the RNA samples were taken.

Population level variation in effectiveness of cancer-relevant immunosurveillance could also relate directly to cancer susceptibility. We reasoned that patients whose MHC genotype could present a larger fraction of driver mutations to the immune system would be more resistant to developing cancer. As homozygous genotype at MHC alleles could reduce the diversity of presented peptides, we compared presentation across patients with different levels of homozygosity. We quantified coverage of cancer causing mutations as the fraction of the 1,018 driver mutations that could be presented by the MHC-II genotype of each patient (STAR Methods) and henceforth refer to this fraction as MHC-II coverage. As expected, patients with more homozygous MHC-II alleles were able to present a smaller fraction of the space due to their decreased MHC diversity (FIG. 6A). MHC-I (using a PHBR-I cutoff of 2) showed a similar trend (FIG. 6B).

Next, we asked whether higher MHC coverage could delay the development of cancer. We reasoned that if two patients acquired a cancer-driving mutation at the same time, the patient with higher MHC coverage would be more likely to expose their mutation to the immune system and stop expansion of the cancer. Thus, high MHC coverage should lead to diagnosis with cancer later in life and vice-versa (FIG. 6C). First, we tested MHC-II, but found no relationship between age at diagnosis and coverage (p=0.51, FIG. 13A). In contrast, patients with higher MHC-I coverage of driver mutations were more often diagnosed with cancer at a later age (p=0.01, controlling for tumor type and ancestry, FIG. 6D). Across tumor types, the 5% of patients with the highest MHC-I coverage were diagnosed with cancer four years later than the 5% of patients with the lowest coverage (p=0.004, FIG. 13B), versus a two-year difference when the highest and lowest 10% was used (p=0.02). Across tumor types, hepatocellular carcinoma showed the most significant difference after multiple testing correction and was diagnosed on average seven years earlier when MHC-I coverage was low. Although coverage of driver and passenger mutations was strongly correlated (MHC-I Pearson's r=0.79, MHC-II Pearson's r=0.68), the significant association with age at diagnosis with MHC-I coverage was not observed for passengers (p=0.11). Within tumor types, MHC-I coverage did not correlate with overall mutation burden (FIG. 13C). These findings suggest that the effect on age is specific to MHC-I coverage of driver mutations rather than to effects of coverage on mutagenesis in general. Using the number of homozygous MHC-I genes in place of coverage showed the same association with age at diagnosis but was more granular because patients fall into discrete bins of homozygous genes counts (p=0.024). The observation that MHC-I, but not MHC-II, coverage is correlated with age at diagnosis supports a protective role for CD8+-driven cytotoxicity. The lack of association with MHC-II suggests that MHC-II-driven CD4+ effector responses against key driver mutations are weaker than CD8+ responses. In addition, either the regulatory role of CD4+-driven immune responses does not depend on coverage of driver mutations or, as indicated in FIG. 2, low variance in interpatient coverage by MHC-II causes this effect to be undetectable.

Part B—Strength of Immune Selection in Tumors Varies with Sex and Age

### Example 27—Data Acquisition

Data were obtained from publicly available sources including The Cancer Genome Atlas (TCGA) Research Network (cancergenome.nih.gov on the World Wide

Web). TCGA normal exome sequences and TCGA clinical data were downloaded from the GDC. Furthermore, TCGA somatic mutations were accessed from the NCI Genomic Data Commons (portal.gdc.cancer.gov/ on the World Wide Web).

### Example 28—Validation Cohort

dbGaP studies (accession numbers: phs001493.v1.p1.c2, phs001041.v1.p1.c1, phs001425.v1.p1.c1, phs001493.v1.p1.c1, phs000980.v1.p1.c1, phs001469.v1.p1.c1, phs000452.v2.p1.c1, phs001451.v1.p1.c1, phs001519.v1.p1.c1, phs001565.v1.p1.c1) were obtained from the dbGaP database and WXS/WGS data obtained from the Sequence Read Archive (SRA) (Leinonen et al., 2010, Nuc. Acids Res., 39:E19-21). Somatic mutation files were obtained from the respective papers associated with each study. Additional non-TCGA patients' WXS/WGS data was obtained from the ICGC and somatic mutation data from the ICGC DCC Data Release (PCAWG and THCA-SA) (Appendix B). The validation cohort's MHC-I and -II genotypes were typed using HLA-HD (Kawaguchi et al., 2017, Hum. Mutat., 38:788:97), and PHBR scores calculated using the method described in “Presentation score assignment”.

### Example 29—HLA Typing

HLA genotyping was performed for class I genes HLA-A, HLA-B, HLA-C and class II genes HLA-DRB1, HLA-DPA1, HLA-DPB1, HLA-DQA1 and HLA-DQB1, which encode three protein determinants of MHC-I peptide binding specificity, HLA DR, HLA-DP, and HLA-DQ. TCGA samples were typed with Polysolver (Shukla et al., 2015, Nat. Biotechnol., 33:1152-1158), with default parameters, for class I and typed with HLA-HD (Kawaguchi et al., 2017, Hum. Mutat., 38:788-97), using default parameters, for class II. Both tools requires germline (whole blood or tissue matched) whole exome sequenced samples. Samples with very low coverage on specific genes are left untyped by HLA-HD. Patients were assigned an HLA-DR type if they were successfully typed for HLA-DRB1. Patients were assigned HLA-DP and -DQ types if they had successful typing for HLA-DPA1/HLA-DPB1 and HLA-DQA1/HLA-DQB1, respectively. Class I and class II types were validated by xHLA (Xie et al., 2017, PNAS USA, 114:8059-64), run with default parameters, and only patients where all alleles agreed in both classes were included in the analysis.

### Example 30—Presentation Score Assignment

Patient presentation scores, as defined in (Marty et al., 2017, Cell, 171:1272-83), were used to represent a particular patient's ability to present a residue given their distinct set of HLA types. For class I, 6 HLA alleles were considered (HLA-A, HLA-B and HLA-C). For class II, 12 HLA-encoded MHC-II molecules (4 combinations of HLA-DPA1/DPB1 and HLA-DQA1/DQB1; 2 alleles of HLA-DRB1 considered twice each—since HLA-DRA1 is invariant—for consistency between resulting molecules). The Patient Harmonic-mean Best Rank (PHBR) score was assigned as the harmonic mean of the best residue presentation scores for each group of MHC-I and MHC-II molecules. A lower patient presentation score indicates that the patient's MHC molecules are more likely to present a residue on the cell surface.

### Example 31—Data Acknowledgements

We would like to thank the TCGA research network for providing data used in the analyses, the ICGC database, as well as the following studies used in the validation cohort.

phs001493.v1.p1.c2 and phs001451.v1.p1.c1 We would also like to thank the Blavatnik Family Foundation, grants from the Broad Institute SPARC program, the National Institutes of Health (NCI-5R01CA155010-02, NHLBI-5R01HL103532-03, NCI-SPORE-2P50CA101942-11A1, NCI-R50-RCA211482A), the Francis and Adele Kittredge Family Immuno-Oncology and Melanoma Research Fund, the Faircloth Family Research Fund, and the DFCI Center for Cancer Immunotherapy Research fellowship and Leukemia and Lymphoma Society.

phs001041.v1.p1.c1 We thank Martin Miller at Memorial Sloan Kettering Cancer Center (MSKCC) for his assistance with the NetMHC server, Agnes Viale and Kety Huberman at the MSKCC Genomics Core, Annamalai Selvakumar and Alice Yeh at the MSKCC HLA typing laboratory for their technical assistance, and John Khoury for assistance in chart review.

phs001425.v1.p1.c1 Christine N. Spencer, Pei-Ling Chen, Michael T. Tetzlaff, Michael A. Davies, Jeffrey E. Gershenwald, Sapna P. Patel, Adi Diab, Isabella C. Glitza, Hussein Tawbi, Alexander J. Lazar, Patrick Hwu, Wen-Jen Hwu, Scott E. Woodman, Rodabe N. Amaria, Victor G. Prieto, and Jennifer A. Wargo enrolled subjects and contributed samples.

phs001493.v1.p1.c1 This study was supported by an AACR KureIt grant.

phs000980.v1.p1.c1 We thank the members of the Thoracic Oncology Service and the Chan and Wolchok labs at MSKCC for helpful discussions, as well as the Immune Monitoring Core at MSKCC, including L. Caro, R. Ramsawak, and Z. Mu, for exceptional support with processing and banking peripheral blood lymphocytes. We thank P. Worrell and E. Brzostowski for help in identifying tumor specimens for analysis. We thank A. Viale for superb technical assistance. We thank D. Philips, M. van Buuren, and M. Toebes for help performing the combinatorial coding screens. This work was supported by the Geoffrey Beene Cancer Research Center (MDH, NAR, TAC, JDW, AS), the Society for Memorial Sloan Kettering Cancer Center (MDH), Lung Cancer Research Foundation (WL), Frederick Adler Chair Fund (TAC), The One Ball Matt Memorial Golf Tournament (EBG), Queen Wilhelmina Cancer Research Award (TNS), The STARR Foundation (TAC, JDW), the Ludwig Trust (JDW), and a Stand Up To Cancer-Cancer Research Institute Cancer Immunology Translational Cancer Research Grant (JDW, TNS, TAC). Stand Up To Cancer is a program of the Entertainment Industry Foundation administered by the American Association for Cancer Research.

phs001469.v1.p1.c1 This work was supported by NIH grants R35CA197633, P01CA168585, 5P50CA168536 and GM08042. A comprehensive description of the data set can be found at PMID:29320474.

phs001519.v1.p1.c1 We thank the Ben and Catherine Ivy Foundation, the Blavatnik Family Foundation, the Broad Institute SPARC program, and NIH (NCI-1R01CA155010-02 (to C.J.W.)), NHLBI-5R01HL103532-03 (to C.J.W.), Francis and Adele Kittredge Family Immuno-Oncology and Melanoma Research Fund (to P.A.O.), Faircloth Family Research Fund (to P.A.O.), NIH/NCI R21 CA216772-01A1 (to D.B.K.), NCI-SPORE-2P50CA101942-11A1 (to D.B.K.); NHLBI-T32HL007627 (to J.B.I.); NCI (R50CA211482) (to S.A. S.), Zuckerman STEM Leadership Program (to I.T.); Benoziyo Endowment Fund for the Advancement of Science (to I.T.); P50 CA165962 (SPORE) and P01 CA163205 (to K.L.L.); DFCI Center for Cancer Immunotherapy Research fellowship (to Z.H.); Howard Hughes Medical Institute Medical Research Fellows Program (to A.J.A.); and American Cancer Society PF-17-042-01-LIB (to N.D.M.). C.J.W. is a scholar of the Leukemia and Lymphoma Society. We thank the Center for Neuro-Oncology, J. Russell and Dana-Farber Cancer Institute (DFCI) Center for Immuno-Oncology (CIO) staff; B. Meyers, C. Harvey and S. Bartel (Clinical Pharmacy); M. Severgnini, K. Kleinsteuber and E. McWilliams, (CIO laboratory); M. Copersino (Regulatory Affairs); T. Bowman (DFHCC Specialized Histopathology Core Laboratory); A. Lako (CIO); M. Seaman and D. H. Barouch (BIDMC); the Broad Institute's Biological Samples, Genetic Analysis and Genome Sequencing Platforms; J. Petricciani and M. Krane for regulatory advice; B. McDonough (CSBio), I. Javeri and K. Nellaiappan (CuriRx) for peptide development.

phs001565.v1.p1.c1 The research reported in this article was supported by BroadIgnite, BroadNext10, NIH K08CA188615, the Howard Hughes Medical Institute, and Stand Up To Cancer—American Cancer Society Lung Cancer Dream Team Translational Research Grant (grant number: SU2C-AACR-DT17-15). Stand Up To Cancer is a program of the Entertainment Industry Foundation. Research grants are administered by the American Association for Cancer Research, the scientific partner of SU2C.

### Example 32—Set of Driver Mutations

Somatic mutations were considered to be recurrent and oncogenic if they occurred in one of the 100 most highly ranked oncogenes or tumor suppressors described by Davoli et al. (2013, Cell, 155:948-62) and were observed in at least 3 TCGA samples. Among these, only mutations that would result in predictable protein sequence changes that could generate neoantigens, including missense mutations and inframe indels, were retained. A total of 1,018 mutations (512 missense mutations from oncogenes, 488 missense mutations from tumor suppressors, 11 indels from oncogenes and 7 indels from tumor suppressors) were obtained (Marty et al., 2017, Cell, 171:1272-83).

### Example 33—Modeling the Effects of PHBR Score on Mutation Probability

Two matrices, for PHBR-I scores and PHBR-II scores, were built from the 1,018 mutations and the 1,912 patients with both PHBR-I and -II calls. Next, a binary mutation matrix yᵢⱼ e {0,1} indicating whether patient i has a specific mutation j was built. The relationship between this binary matrix, the matched 1,912×1,018 matrices with log PHBR-I and -II scores, x1ᵢⱼ and x2ᵢⱼ, respectively, and the variable of interest (sex or age) for patient i and mutation j were evaluated. A generalized additive model was fit for the centered log PHBR-I, centered log PHBR-II scores, centered sex (coded 0/1 for males/females) or centered age, and mutation probability with the GAM function in the MGCV R package (Wood et al., 2001, R. news, 1:20-5). To estimate the effects of PHBR and sex or age on probability of mutation, the following random effects models were considered:

Logit(P(yᵢⱼ=1))=β₁x1ᵢⱼ+β₂x2ᵢⱼ+β₃Sexᵢ+β₁x1ᵢⱼ*Sexᵢ+β₂x2ᵢⱼ*Sexᵢ+ηᵢ

Logit(P(yᵢⱼ=1))=β₁x1ᵢⱼ+β₂x2ᵢⱼ+β₃Ageᵢ+β₁x1ᵢⱼ*Ageᵢ+β₂x2ᵢⱼ*Ageᵢ+ηᵢ

And a PHBR-II specific model (results in Table 4):

Logit(P(yᵢⱼ=1))=β₁x2ᵢⱼ+β₂Ageᵢ+β₂Sexᵢ+β₂x2ᵢⱼ*Sexᵢ+β₂x2ᵢⱼ*Ageᵢ+ηᵢ

where ηᵢ˜N(0, θ_{η}) are random effects capturing different mutation propensities among patients. In these models, βₙ measures the effect of the log-PHBR-I, log-PHBR-II, and sex or age. This analysis was repeated for the validation cohort.

TABLE 4
Quantitative estimate of the association between PHBR-II score
and mutation occurrence in sex- and age-specific TCGA cohorts

| Parametric coefficients | Estimate | Pr(>\|z\|) |
|---|---|---|
| PHBR-II | 0.31 | <2e−16 |
| Sex | −0.05 | 0.24 |
| Age | −0.002 | 0.16 |
| PHBR-II: Sex | 0.12 | 0.005 |
| PHBR-II: Age | −0.003 | 0.01 |

### Example 34—Mutational Signature Analysis

Mutational signatures analysis was performed using a previously developed computational framework SigProfiler (Alexandrov et al., 2013, Cell Rep., 3:246-59). A detailed description of the workflow of the framework can be found in (Alexandrov et al., 2013, Cell Rep., 3:246-59; biorxiv.org/content/early/2018/05/15/322859 on the World Wide Web), while the code can be downloaded freely from mathworks.com/matlabcentral/fileexchange/38724-sigprofiler on the World Wide Web).

### Example 35—Statistical Analysis

All boxplots were evaluated using the default one-tailed Mann Whitney U statistical test, via the scipy.stats Python package. Mutational signature sex-specific distributions were also compared using the one-tailed Mann Whitney U test, and p-values were adjusted using the Benjamin-Hochberg Procedure.

### Example 36—Code Availability

Code to reproduce findings and figures can be freely accessed at github.com/CarterLab/HLA-immunoediting on the World Wide Web.

### Example 37—Results

A set of 1,018 driver mutations, defined in (Marty et al., 2017, Cell, 171:1272-83), were examined, since driver mutations are more persistent in the clonal architecture of an individual's cancer and confer a selective growth advantage. MHC-I and MHC-II types were assigned based on the consensus of two exome-based calling methods (Shukla et al, 2015, Nat. Biotechnol., 33:1152-8; Xie et al., 2017, PNAS USA, 114:8059-64; and Kawaguchi et al., 2017, Hum. Mutat., 38:788-97) and only microsatellite-stable (MSS) TCGA patients that had identically matched typing were considered. Ultimately, 2,554 patients with confident MHC-I calls and 2,681 patients with confident MHC-II calls who were diverse in sex, with more males than females (FIG. 19A), and a broad distribution of age at diagnosis (FIG. 19B) were analyzed. Patients were categorized into subgroups according to sex (male versus female) and age (younger versus older based on 30th and 70th percentiles at age of diagnosis). All MHC-I and MHC-II cohorts had a similar average number of driver mutations (FIG. 20). It was previously found that TCGA patients with somatic MHC-I mutations had altered mutational landscapes, with a higher fraction of binding neoantigens than patients without MHC-I mutations (Wong et al., 2011, Bioinformatics, 27:2147-8). To ensure that somatic MHC-I mutations would not skew the driver mutation PHBR-I score distributions, scores for patients with and without MHC-I mutations grouped by sex and age were compared and no significant differences were found (FIG. 21). PHBR scores were used to predict patients' potential to present the set of 1,018 driver mutations, then the distribution of PHBR-I and PHBR-II scores and the fraction of presentable driver mutations between the sex- and age-specific groups were compared and no significant difference were found (FIG. 22A-22F). The overall similarity of MHC presentation suggests that patients of both sexes and various ages at diagnosis present driver mutations with roughly equivalent efficacy, implying that specificity of MHC presentation resulting from inherited combinations of alleles is not the mechanism causing differences in immune checkpoint inhibitors (ICPi) response rate.

It was reasoned that the discrepancy might be due to differences in the strength of immune selection, e.g., tumors with stronger immunoediting should retain fewer driver mutations that are presentable to T cells by the patient's own MHC molecules. For sex- and age-specific groups in each cohort, the PHBR-I and PHBR-II score distributions for expressed driver mutations observed in patient tumors were compared. Across pan-cancer cohorts, females were at a significant disadvantage in presenting their driver mutations by both their MHC-I and MHC-II molecules (FIG. 14A-14B, p<2.8e-04 and p<8.7e-05, respectively). Younger patients also tended to have worse presentation of driver mutations by both MHC-I and MHC-II molecules (FIG. 14C-14D, p<0.02 and p<3.5e-05, respectively). These differences suggest that tumors in female and younger patients undergo greater immunoediting than those in male and older patients.

Next, the immune system's ability to eliminate effectively-presented mutations was explored. Sex- and age-specific generalized additive models with random effects were used to account for variation in mutation rate across individuals and examined the coefficients corresponding to independent and interaction effects for PHBR-I, PHBR-II, and sex or age to assess their contribution to immune selection. In both models, it was found that PHBR-I and PHBR-II scores alone had significant effects on the probability of a mutation to be a target of immune selection (Table 5). Positive coefficients for both PHBR scores indicate that the higher the PHBR score (i.e., poorer presentation), the higher the probability of mutation. Furthermore, when the influence of both scores on probability of mutation were quantified using odds ratios between respective 25th and 75th percentiles, it was found that PHBR-II (OR: 2.11, CI [2.01, 2.20]) has a much larger impact on probability of mutation than PHBR-I (OR: 1.25, CI [1.23, 1.27]), echoing the larger effect sizes seen in FIG. 14. As expected, sex and age alone did not influence the probability of mutation; however, of particular interest are the interaction terms that indicate the influence of PHBR scores within the context of sex and age. While the PHBR-I:sex and PHBR-I:age interactions did not reach significance, the PHBR-II:sex and PHBR-II:age interactions were significant. The negative PHBR-II:age estimate indicates a stronger effect of PHBR-II contribution to the probability of mutation in younger patients. On the other hand, positive PHBR-II:sex estimate indicates a stronger effect of PHBR-II contributing to probability of mutation in females according to the model formulation. Collectively, these results suggest stronger immunoediting in females and younger patients.

TABLE 5
Quantitative estimate of the association between PHBR score and
mutation occurrence in sex- and age-specific cohorts. Estimates and
p-values are shown for a generalized additive model with random
effects relating PHBR scores to the set of expressed driver mutations
observed ≥2 times in this cohort

| | Parametric coefficients | Estimate | Pr(>\|z\|) |
|---|---|---|---|
| Sex analysis | PHBR-I | 0.095 | 3.68e−07 |
| | PHBR-II | 0.28 | <2e−16 |
| | Sex | −0.046 | 0.32 |
| | PHBR-I: Sex | 0.04 | 0.29 |
| | PHBR-II: Sex | 0.12 | 0.013 |
| Age analysis | PHBR-I | 0.095 | 2.86e−07 |
| | PHBR-II | 0.29 | <2e−16 |
| | Age | −0.0025 | 0.09 |
| | PHBR-I: Age | −0.0011 | 0.35 |
| | PHBR-II: Age | −0.0043 | 0.005 |

As females and younger patients both demonstrated stronger immunoediting compared to males and older patients, the cohorts were further segregated simultaneously by sex and age, and the distribution of PHBR-I and -II scores were investigated for these groups. It was found that sex and age effects are cumulative, with tumors in younger females exhibiting significantly higher selective pressure by MHC than those in the other three groups (FIG. 15). A profound difference between PHBR score distributions for younger females and older males was noticed. Because younger males had worse MHC-II presentation of their driver mutations compared to older females, we sought to ensure that sex had an effect on immunoediting independent of age. In a model incorporating sex, age, and PHBR-II scores, both PHBR-II:sex and PHBR-II:age were independently significant (Table 4). These results demonstrate that more aggressive immunoediting in younger females selects for tumors with driver mutations that are less visible to the immune system.

It was next explored whether sex- and age-specific effects could be driven by differences in environmental exposure rather than the strength of immunoediting. Mutational signatures assign specific mutations to different mutagenic processes, allowing the exploration of differences in environmental exposure across sex and age. The sex-specific occurrence of mutational signatures were compared in each tumor type and only a minority of instances were found where signature strength was weakly but significantly associated with sex (FIG. 16A). Importantly, only four of the signatures where sex-specific differences were observed contribute to the set of driver mutations used for this analysis (FIG. 16B), suggesting a very low impact of environmental exposures on sex-specific effects on immunoediting. Indeed, when the tumor types with significant signature differences were excluded, sex- and age-related differences in immunoediting were still observed (Table 6). In addition, only two signatures correlated with age, both of which have known association with aging (Alexandrov et al., 2015, Nat. Genet., 47:1402-7). C>T and T>C mutations were examined, which are hallmarks of signature 01 and 05, respectively, and it was found that observed driver mutations in these categories were broadly distributed across age at diagnosis. To explain weaker immunoediting in older individuals, age-related mutations would have to be better presented (have lower PHBR scores) than other mutations. Instead, it was found that C>T and T>C mutations were significantly more poorly presented (had slightly higher PHBR scores) than other mutations across all possible MHC-I and MHC-II alleles, suggesting that these mutations, and by extension, signatures 01 and 05, could not drive the apparent age-associated difference in immunoediting (FIG. 16C). Thus, it was concluded that the sex- and age-specific effects on immunoediting are not likely due to exposure differences (Alexadrov et al., 2013, Nature, 500:415-21; Alexandrov et al., 2015, Nat. Genet., 47:1402-7).

TABLE 6
Quantitative estimate of the association between PHBR score and
mutation occurrence in sex- and age-specific TCGA cohorts, without
tumor types significantly associated with sex-specific mutational
signature ratios. Estimates and p-values are shown for a generalized
additive model with random effects relating PHBR scores to set of
driver mutations observed ≥ times in the TCGA cohort

| | Parametric coefficients | Estimate | Pr(>\|z\|) |
|---|---|---|---|
| Sex analysis | PHBR-I | 0.15 | 1.80e−10 |
| | PHBR-II | 0.30 | <2e−16 |
| | Sex | −0.06 | 0.23 |
| | PHBR-I: Sex | 0.04 | 0.23 |
| | PHBR-II: Sex | 0.10 | 0.07 |
| Age analysis | PHBR-I | 0.15 | 1.21e−10 |
| | PHBR-II | 0.31 | <2e−16 |
| | Age | −0.002 | 0.28 |
| | PHBR-I: Age | −0.0025 | 0.086 |
| | PHBR-II: Age | −0.0047 | 0.01 |

We sought validation of these findings in a cohort of 465 MHC-I typed patients and 426 MHC-II typed patients, compiled from published dbGaP studies and non-TCGA samples in the International Cancer Genome Consortium (ICGC) database (Zhang et al., 2011, Database, bar026) and filtered to exclude tumor types not represented in TCGA. While fewer tumor types were represented relative to the discovery cohort, these patients were diverse with respect to sex and age at diagnosis, with slightly more males than females, and similar average numbers of driver mutations and PHBR score distributions for all patient groups (FIG. 23). To maximize the number of samples available, expression data for the validation cohort was not required. To account for this limitation, it was verified that previous TCGA results remain without requiring driver mutations to be expressed (FIG. 24, Table 7).

TABLE 7
Quantitative estimate of the association between PHBR score and
mutation occurrence in sex and age-specific TCGA cohorts, without
filtering mutations based on expression. Estimates and p-values are shown
for a generalized additive model with random effects relating PHBR
scores to set of driver mutations observed ≥2 times in the TCGA cohort

| | Parametric coefficients | Estimate | Pr(>\|z\|) |
|---|---|---|---|
| Sex analysis | PHBR-I | 0.074 | 2.05e−05 |
| | PHBR-II | 0.27 | <2e−16 |
| | Sex | −0.064 | 0.16 |
| | PHBR-I: Sex | 0.036 | 0.31 |
| | PHBR-II: Sex | 0.13 | 0.0038 |
| Age analysis | PHBR-I | 0.076 | 1.37e−05 |
| | PHBR-II | 0.27 | <2e−16 |
| | Age | −0.0017 | 0.24 |
| | PHBR-I: Age | −0.0011 | 0.32 |
| | PHBR-II: Age | −0.0045 | 0.002 |

It was found, as in the discovery cohort, that driver mutations had significantly poorer MHC-II presentation in younger females compared to older females and older males (p<2.16e-05, p<0.001), and trended toward significance relative to younger males (p<0.29) (FIG. 17F). While the trends did not reach significance for MHC-I (FIG. 17E), the linear model analysis in the discovery cohort suggested that the effects of age and sex were mediated predominantly by MHC-II (Table 5). When evaluating PHBR score distributions in groups separated by sex and age, only PHBR-II was significantly different between younger and older patients (FIG. 17A, 17B, 17C, 17D). It was noted that PHBR score distributions varied between the discovery and validation cohort for the four groups (FIG. 25), with stronger effects of age potentially masking more subtle sex-specific effects within the sample sizes available. In the validation set, younger males had significantly poorer MHC-II presentation of driver mutations than both older males (p<0.02) and older females (p<0.001). The sex- and age-specific analyses were repeated using the generalized additive models and it was found that, for both sex and age, PHBR scores significantly influence the probability of mutation, with higher PHBR scores (i.e., worse presentation) leading to higher probability of mutation (Table 8). In addition, significant PHBR-I:sex and PHBR-II:age interaction coefficients show that female sex and younger age, in combination with PHBR score, have stronger effects on probability of mutation.

TABLE 8
Quantitative estimate of the association between PHBR score and
mutation occurrence in sex and age-specific validation cohorts.
Estimates and p-values are shown for a generalized additive model
with random effects relating PHBR scores to set of driver mutations
observed in the validation cohort

| | Parametric coefficients | Estimate | Pr(>\|z\|) |
|---|---|---|---|
| Sex analysis | PHBR-I | 0.098 | 0.008 |
| | PHBR-II | 0.15 | 0.0006 |
| | Sex | 0.22 | 0.015 |
| | PHBR-I: Sex | 0.18 | 0.01 |
| | PHBR-II: Sex | 0.008 | 0.92 |
| Age analysis | PHBR-I | 0.076 | 0.007 |
| | PHBR-II | 0.27 | 0.005 |
| | Age | −0.0017 | 0.06 |
| | PHBR-I: Age | −0.0011 | 0.34 |
| | PHBR-II: Age | −0.0045 | 0.0035 |

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A computer implemented method for determining whether a subject is at risk of having or developing a cancer, the method comprising:
a) genotyping the subject's major histocompatibility complex class II (MHC-II); and
b) scoring the ability of the subject's MHC-II to present a mutant cancer-associated peptide based upon a library of known cancer-associated peptide sequences derived from subjects, wherein the produced score is the MHC-II presentation score; wherein:
i) if the subject is a poor MHC-II presenter of specific mutant cancer-associated peptides, the subject has an increased likelihood of having or developing the cancer for which the specific mutant cancer-associated peptides are associated; or
ii) if the subject is a good MHC-II presenter of specific mutant cancer-associated peptides, the subject has a decreased likelihood of having or developing the cancer for which the specific mutant cancer-associated peptides are associated.

2. The method of claim 1, further comprising:
c) determining whether a biopsy sample obtained from the subject comprises DNA encoding a mutant cancer-associated peptide based upon a library of cancer-associated mutations obtained from subjects.

3. The method of claim 2, wherein the biopsy sample is a liquid biopsy sample.

4. The method of claim 3, wherein the liquid biopsy sample is blood, saliva, urine, or other body fluid.

5. The method of claim 2, wherein the library of cancer-associated mutations is obtained by whole genome sequencing of subjects.

6. The method of claim 1, wherein the step of scoring the ability of the subject's MHC-II to present a mutant cancer-associated peptide comprises using a predicted MHC-II affinity for a given mutation xij, where x is the MHC-II affinity of subject i for mutation j to fit a mixed-effects logistic regression model that follows a model equation obtained from a large dataset of subjects from which MHC-II genotypes and presence of peptides of interest can be obtained:
logit(P(yᵢⱼ=1|xᵢⱼ))=ηⱼ+γ log(xᵢⱼ)
wherein:
yᵢⱼ is a binary mutation matrix yᵢⱼ ∈{0,1} indicating whether a subject i has a mutation j;
xᵢⱼ is a binary mutation matrix indicating predicted MHC-II binding affinity of subject i having mutation j;
γ measures the effect of the log-affinities on the mutation probability; and
ηj˜N(0, ϕ_{η}) are random effects capturing residue-specific effects,
wherein the model tests the null hypothesis that γ=0 and calculates odds ratios for MHC-II affinity of a mutation and presence of a cancer.

7. The method of claim 6, wherein the predicted MHC-II affinity for a given mutation xᵢⱼ is a Subject Harmonic-mean Best Rank (PHBR) score.

8. The method of claim 7, wherein the PHBR score is obtained by aggregating MHC-II binding affinities of a set of mutant cancer-associated peptides by referring to a pre-determined dataset of peptides binding to MHC-II molecules encoded by at least 12 different HLA alleles.

9. The method of claim 8, wherein the mutant cancer-associated peptide contains an amino acid substitution, and wherein the set of peptides consists of at least 15 of all possible 15-amino acid long peptides incorporating the substitution at every position along the peptide.

10. The method of claim 8, wherein the mutant cancer-associated peptide contains an amino acid insertion or deletion, and wherein the set of peptides consists of at least 15 of all possible 15-amino acid long peptides incorporating the insertion or deletion at every position along the peptide.

11. The method according to claim 1, wherein the set of mutant cancer-associated peptides comprises any one or more of the mutations shown in Appendix A, wherein the presence of any one of these mutations indicates the presence of or increased risk of developing cancer.

12. The method according to claim 1, wherein the cancer is a bladder urothelial carcinoma (BLCA), a breast invasive carcinoma (BRCA), a colon adenocarcinoma (COAD), a glioblastoma multiforme (GBM), a head and neck squamous cell carcinoma (HNSC), a brain lower grade glioma (LGG), a liver hepatocellular carcinoma (LIHC), a lung adenocarcinoma (LUAD), lung squamous cell carcinoma (LUSC), an ovarian serous cystadenocarcinoma (OV), a pancreatic adenocarcinoma (PAAD), a prostate adenocarcinoma (PRAD), a rectum adenocarcinoma (READ), a skin cutaneous melanoma (SKCM), a stomach adenocarcinoma (STAD), a thyroid carcinoma (THCA), a uterine corpus endometrial carcinoma (UCEC), or a uterine carcinosarcoma (UCS).

13. A computing system for determining whether a subject is at risk of having or developing a cancer, the system comprising:
a) a communication system for using a library of cancer-associated peptides derived from subjects; and
b) a processor for scoring the ability of the subject's major histocompatibility complex class II (MHC-II) to present a mutant cancer-associated peptide based upon a library of cancer-associated peptides derived from subjects,
wherein the produced score is the MHC-II presentation score.

14. The computing system according to claim 13, wherein the step of scoring the ability of the subject's MHC-II to present a mutant cancer-associated peptide comprises using a predicted MHC-II affinity for a given mutation xij, where x is the MHC-II affinity of subject i for mutation j to fit a mixed-effects logistic regression model that follows a model equation obtained from a large dataset of subjects from which MHC-II genotypes and presence of peptides of interest can be obtained:
logit(P(yij=1|xij))=ηj+γ log(xij)
wherein:
yij is a binary mutation matrix yij ∈{0,1} indicating whether a subject i has a mutation j;
xij is a binary mutation matrix indicating predicted MHC-II binding affinity of subject i having mutation j;
γ measures the effect of the log-affinities on the mutation probability; and
ηj˜N(0, ϕη) are random effects capturing residue-specific effects,
wherein the model tests the null hypothesis that γ=0 and calculates odds ratios for MHC-II affinity of a mutation and presence of a cancer.

15. The computing system according to claim 14, wherein the predicted MHC-II affinity for a given mutation xij is a Subject Harmonic-mean Best Rank (PHBR)-II score.

16. The computing system according to claim 14, wherein the PHBR-II score is obtained by aggregating MHC-II binding affinities of a set of mutant cancer-associated peptides by referring to a pre-determined dataset of peptides binding to MHC-II molecules encoded by at least 12 different HLA alleles.

17. The computing system according to claim 16, wherein the mutant cancer-associated peptide contains an amino acid substitution, and wherein the set of peptides consists of at least 15 of all possible 15-amino acid long peptides incorporating the substitution at every position along the peptide.

18. The computing system according to claim 16, wherein the mutant cancer-associated peptide contains an amino acid insertion or deletion, and wherein the set of peptides consists of at least 15 of all possible 15-amino acid long peptides incorporating the insertion or deletion at every position along the peptide.
